(19) United States
(12) Patent Application Publication
(10) Pub. No.: US 2026/0354836 A1
(43) Pub. Date: Nov. 12, 2026

(54) RECIPROCATING SAW BLADE SYSTEM AND METHODS OF MAKING AND USING THE SAME

(71) Applicant: Restor3D, Inc., DURHAM, NC (US)

(72) Inventors: Deepak Padmanabhan, Cary, NC (US); Nicholas James Heydinger, Durham, NC (US)

(21) Appl. No.: 19/668,357

(22) Filed: May 5, 2026

Related U.S. Application Data
(60) Provisional application No. 63/800,972, filed on May 6, 2025.

(51) Int. Cl. A61B 17/14; A61F 2/46; A61B 17/00; A61B 17/56

(52) U.S. Cl. CPC A61B 17/144; A61F 2/4606; A61B 2017/0046; A61B 2017/00738; A61B 2017/564

(57) ABSTRACT

A saw blade for a reciprocating saw is provided, the saw blade comprising: a saw interface portion comprising a first planar surface, a first edge, a second edge, a support rib extending perpendicularly from the first planar surface, and a first axis that is coaxial with an axis of reciprocation of the reciprocating saw; an offset portion extending from the first edge at an angle between about 90° and 180° relative to the first edge, and comprising a second planar surface that is co-planar with the first planar surface; a cutting portion extending from the offset portion and that comprises a third planar surface extending at an angle between about 0° and 90° relative to the second planar surface; and a plurality of cutting teeth disposed along an edge of the cutting portion, wherein the third planar surface is substantially parallel to the axis of reciprocation.

### CROSS-REFERENCES TO RELATED APPLICATIONS

This application claims the benefit of priority to U.S. Provisional Patent Application No. 63/800,972, filed May 6, 2025, entitled “RECIPROCATING SAW BLADE SYSTEM AND METHODS OF MAKING AND USING THE SAME,” of which is incorporated herein by reference in its entirety.

### BACKGROUND

Joint replacement surgery involves replacement of an existing joint section in a patient with an artificial joint implant that includes some or all parts of a particular joint and/or surrounding sections of bones. In the case of ankle arthroplasty, or total ankle replacement (TAR) surgery, certain aspects of the ankle joint are removed and replaced with prosthetic components that may simulate natural ankle movement. Further, TAR surgery may be an alternative to an ankle fusion approach (e.g., arthrodesis) because it may preserve the functional range of motion of the ankle, which may lead to better patient health and satisfaction.

Joint replacement surgery often requires resection of portions of the joint. In some instances, accessing portions of the joint for resection may be challenging, resulting in invasive surgical techniques. Surgeons use a variety of tools to perform resection including, but not limited to, drills, broaches, and saws. Each type of tool used in joint replacement surgery has certain advantages and limitations. Particularly where access to tissue is challenging, tools like drills and saws are limited by their size and configuration and may require multiple tools to be used to achieve desired resection.

As such, there is a need for improved systems and methods for resecting tissue in tight spaces.

### BRIEF SUMMARY OF THE DISCLOSURE

The following embodiments and aspects thereof are described and illustrated in conjunction with systems, devices, and processes that are meant to be exemplary and illustrative, not limiting in scope.

Briefly described, aspects of the present disclosure generally relate to a reciprocating saw blade having a cutting portion offset from the axis of reciprocation of a saw, as well as processes for making and using the same. Additionally, the present disclosure relates to saw blades used in joint replacement surgeries that may include patient-specific components.

According to a first aspect, the present disclosure relates to a saw blade for a reciprocating surgical saw, the reciprocating surgical saw having an axis of reciprocation, the saw blade comprising: a first end and a second end; a saw interface portion at the first end, the saw interface portion comprising: a first surface; a first edge; a second edge; and a first axis, wherein the first axis is coaxial with an axis of reciprocation of a reciprocating surgical saw, wherein the saw interface portion is configured to be removably coupled to a drive component of the reciprocating surgical saw; an offset portion extending from the first edge, wherein the offset portion extends from the first edge at an offset angle between about 90° and 180° relative to the first edge; the offset portion comprising a second surface, the second surface substantially co-planar with the first surface; a cutting portion extending from the offset portion, wherein the cutting portion comprises a third surface, the third surface extending at a cutting angle between about 0° and 90° relative to the second surface; and a plurality of cutting teeth disposed along at least one edge of the cutting portion, wherein the third surface is substantially parallel to the axis of reciprocation.

According to a second aspect, the saw blade of the first aspect, or any other aspect, wherein the saw blade comprises a metallic material.

According to a third aspect, the saw blade of the first aspect, or any other aspect, further comprising at a first collar engagement tab extending from the first edge of the saw interface portion.

According to a fourth aspect, the saw blade of the third aspect, or any other aspect, further comprising a second collar engagement tab extending from the second edge of the saw interface portion.

According to a fifth aspect, the saw blade of the first aspect, or any other aspect, wherein the saw interface portion further comprises a retention feature.

According to a sixth aspect, the saw blade of the fifth aspect, or any other aspect, wherein the retention feature is a pocket that is recessed into the first surface.

According to a seventh aspect, the saw blade of the fifth aspect, or any other aspect, wherein the retention feature is a first support rib extending perpendicularly from a first side of the first surface.

According to an eighth aspect, the saw blade of the seventh aspect, or any other aspect, wherein the retention feature further comprises a second support rib extending perpendicularly from a second side of the first surface.

According to a ninth aspect, the saw blade of the fifth aspect, or any other aspect, wherein the retention feature is a through-hole opening.

According to a tenth aspect, the present disclosure relates to a method for resecting bone tissue, the method comprising: making an incision to access a patient’s bone tissue; obtaining a reciprocating surgical saw, the reciprocating surgical saw comprising: an axis of reciprocation; and a blade collar; obtaining a saw blade for the reciprocating surgical saw, the saw blade comprising: a saw interface portion comprising: a first planar surface; a first edge; a second edge; and a first axis that is coaxial with the axis of reciprocation; an offset portion extending from the first edge, the offset portion comprising a second planar surface, the second planar surface being co-planar with the first planar surface, wherein the offset portion extends from the first edge at an offset angle between about 90° and 180° relative to the first edge; a cutting portion extending from the offset portion, wherein the cutting portion comprises a third planar surface, the third planar surface extending at a cutting angle between about 0° and 90° relative to the second planar surface; a plurality of cutting teeth disposed along at least one edge of the cutting portion; and wherein the third planar surface is substantially parallel to the axis of reciprocation; inserting the saw interface portion into the blade collar and securing the saw blade to the reciprocating surgical saw; and resecting a portion of the bone tissue using the reciprocating surgical saw.

According to an eleventh aspect, the method of the tenth aspect, or any other aspect, further comprising the steps of: positioning a resection guide on a bone surface, the resection guide including a receiving feature for guiding the saw blade; inserting the saw blade through the receiving feature; and activating the reciprocating surgical saw.

According to a twelfth aspect, the method of the eleventh aspect, or any other aspect, wherein the receiving feature is a blade collar.

According to a thirteenth aspect, the method of the eleventh aspect, or any other aspect, wherein the step of positioning the resection guide on the bone surface further includes removably fixing the resection guide to the bone surface.

According to a fourteenth aspect, the method of the tenth aspect, or any other aspect, wherein the saw blade further comprises a retention feature disposed on the first planar surface.

According to a fifteenth aspect, the method of the fourteenth aspect, or any other aspect, wherein the retention feature is provided in form of at least one support rib, the at least one support rib extending perpendicularly from at least one side of the first planar surface.

According to a sixteenth aspect, the method of the fourteenth aspect, or any other aspect, wherein the retention feature is provided in form of a through-hole opening disposed on a portion of the first planar surface.

According to a seventeenth aspect, the method of the fourteenth aspect, or any other aspect, wherein the retention feature is provided in form of a pocket that is recessed onto a portion of the first planar surface.

According to an eighteenth aspect, the present disclosure relates to a method for total ankle replacement (TAR) of an ankle of a patient, wherein the ankle includes a tibia having a medullary cavity, the method comprising: making an anterior incision to the ankle; resecting a portion of the tibia to form a prepared tibial surface; positioning a tibial trial on the prepared tibial surface; removably fixing the tibial trial to the tibia; distracting the ankle to form a workspace within the ankle; inserting a saw blade into the tibial trial, the saw blade removably attached to a reciprocating surgical saw, the saw blade comprising: a saw interface portion comprising: a first surface; a first edge; a second edge; and a first axis, wherein the first axis is coaxial with an axis of reciprocation of a reciprocating surgical saw, wherein the saw interface portion is configured to be removably coupled to a drive component of the reciprocating surgical saw; an offset portion extending from the first edge, the offset portion comprising a second planar surface, the second planar surface being co-planar with the first planar surface, wherein the offset portion extends from the first edge at an offset angle between about 90° and 180° relative to the first edge; a cutting portion extending from the offset portion, wherein the cutting portion comprises a third planar surface, the third planar surface extending at a cutting angle between about 0° and 90° relative to the second planar surface; a plurality of cutting teeth disposed along at least one edge of the cutting portion, wherein the third planar surface is substantially parallel to the axis of reciprocation, and the reciprocating surgical saw comprising: an axis of reciprocation; and a blade collar; resecting the prepared tibial surface with the saw blade to form an intramedullary canal; inserting a guide wire into the medullary cavity; inserting a flexible reamer assembly into the tibial trial; reaming into the medullary cavity to continue forming the intramedullary canal; inserting a stem component of a tibial implant into the intramedullary canal; and attaching a base component of the tibial implant to the stem component.

According to a nineteenth aspect, the method of the eighteenth aspect, or any other aspect, further comprising the steps of: positioning a resection guide in the tibial trial, the resection guide including a retention feature for guiding the saw blade; inserting the saw blade through the retention feature; and activating the reciprocating surgical saw.

According to a twentieth aspect, the method of the eighteenth aspect, or any other aspect, wherein the step of positioning the resection guide in the tibial trial further includes removably securing the resection guide to the tibial trial.

It will be understood by those skilled in the art that one or more aspects of this disclosure can meet certain objectives, while one or more other aspects can lead to certain other objectives. Various modifications to the illustrated embodiments will be readily apparent to those skilled in the art, and the generic principles herein can be applied to other embodiments and applications without departing from embodiments of the disclosure. Other objects, features, benefits, and advantages of the present disclosure will be apparent in this summary and descriptions of the disclosed embodiments, and will be readily apparent to those skilled in the art. Such objects, features, benefits, and advantages will be apparent from the above as taken in conjunction with the accompanying figures and all reasonable inferences to be drawn therefrom.

### BRIEF DESCRIPTION OF THE DRAWINGS

FIG. 1 is a perspective view of an exemplary saw blade, according to one embodiment;

FIG. 2 is another perspective view of the saw blade of FIG. 1;

FIG. 3 is a top view of the saw blade of FIG. 1;

FIG. 4 is a bottom view of the saw blade of FIG. 1;

FIG. 5 is a side view of the saw blade of FIG. 1;

FIG. 6 is another side view of the saw blade of FIG. 1;

FIG. 7 is another side view of the saw blade of FIG. 1;

FIG. 8 is another side view of the saw blade of FIG. 1;

FIG. 9 is a perspective view of another exemplary saw blade, according to one embodiment;

FIG. 10 is another perspective view of the saw blade of FIG. 9;

FIG. 11 is a top view of the saw blade of FIG. 9;

FIG. 12 is a bottom view of the saw blade of FIG. 9;

FIG. 13 is a side view of the saw blade of FIG. 9;

FIG. 14 is another side view of the saw blade of FIG. 9;

FIG. 15 is another side view of the saw blade of FIG. 9;

FIG. 16 is another side view of the saw blade of FIG. 9;

FIG. 17 is a perspective view of another exemplary saw blade, according to one embodiment;

FIG. 18 is another perspective view of the saw blade of FIG. 17;

FIG. 19 is a top view of the saw blade of FIG. 17;

FIG. 20 is a bottom view of the saw blade of FIG. 17;

FIG. 21 is a side view of the saw blade of FIG. 17;

FIG. 22 is another side view of the saw blade of FIG. 17;

FIG. 23 is another side view of the saw blade of FIG. 17;

FIG. 24 is another side view of the saw blade of FIG. 17;

FIG. 25 is a perspective view of another exemplary saw blade, according to one embodiment;

FIG. 26 is another perspective view of the saw blade of FIG. 25;

FIG. 27 is a top view of the saw blade of FIG. 25;

FIG. 28 is a bottom view of the saw blade of FIG. 25;

FIG. 29 is a side view of the saw blade of FIG. 25;

FIG. 30 is another side view of the saw blade of FIG. 25;

FIG. 31 is another side view of the saw blade of FIG. 25;

FIG. 32 is another side view of the saw blade of FIG. 25;

FIG. 33 is a perspective view of another exemplary saw blade, according to one embodiment;

FIG. 34 is a perspective view of another exemplary saw blade, according to one embodiment;

FIG. 35 is a perspective view of an exemplary tibial trial, according to one embodiment;

FIG. 36 is a front view of an exemplary TAR assembly, according to one embodiment;

FIG. 37 is a top view of the TAR assembly of FIG. 36;

FIG. 38 is a side section view of the TAR assembly of FIG. 36;

FIG. 39 is a bottom view of the TAR assembly of FIG. 36;

FIG. 40A is a flowchart of an exemplary method, according to one embodiment; and

FIG. 40B is a continuation of the flowchart of FIG. 40A.

### DETAILED DESCRIPTION

According to some embodiments, this disclosure relates to various embodiments of an exemplary total ankle replacement (TAR) procedure and system. The bones of the ankle joint include the tibia (shinbone), fibula (smaller bone in the lower leg), and talus (bone in the foot). During the TAR procedure, a user (i.e., surgeon) may remove at least a portion of a damaged patient ankle joint, including damaged tibial, fibular, and/or talar surfaces. Generally, the TAR procedure involves making an anterior incision on the patient’s ankle, removing portions of damaged bone and/or cartilage to reveal (at least) a prepared tibial surface, and installing the TAR system into the prepared tibial surface as described in U.S. Non-provisional Patent Application No. 18/627,137 (incorporated herein by reference in its entirety, for all purposes). Thus, the TAR system may replace/mimic native joint movement, improve joint function, relieve pain, and/or enhance overall life quality for patients with degenerative ankle joint conditions.

According to various embodiments, the present disclosure relates to one or more blades for use with a reciprocating saw. The one or more blades may be used as part of the TAR procedure or any other suitable surgical applications. At a high level, the blade may be designed for precise applications and may be advantageous for use within hard to reach joint spaces having minimal clearance (e.g., the ankle). For instance, the blade may be designed to cut and resect bony tissues (e.g., subchondral tibial or talar bone) in a plane that is offset from an axis of reciprocation of the reciprocating saw, wherein the axis of reciprocation is a linear axis of actuation of the drive component of the reciprocating saw. In some embodiments, the cutting plane is substantially parallel to the axis of reciprocation.

According to various embodiments, the one or more blades may be provided in the form of a saw blade. In general, saw blades for use in surgical procedures include features for removably attaching at least one end of the blade to a reciprocating saw, which will be described in general terms below. Saw blades may be planar in nature and include cutting features disposed on at least one edge of the saw blade. In some embodiments, the cutting features include a plurality of teeth arranged in any suitable configuration. Some non-limiting examples of teeth profiles include coarse teeth for making aggressive cuts, fine teeth for making less aggressive cuts, offset teeth, staggered teeth, oscillating teeth, double sided teeth, and angled teeth. In some embodiments, the teeth may have rectangular, U-shaped, or V-shaped edges.

Additionally, in some embodiments, the saw blades may be made from a metallic material (e.g., stainless steel, titanium), a ceramic material, a composite material, or any other suitable material. The saw blades may be designed to be disposable or reusable, depending on user preferences. As will be described herein, the saw blades may be included within a kit as sterile or non-sterile.

Reciprocating saws are well known in the art. One example of a reciprocating surgical saw may include a housing having an elongated barrel section, a pistol-grip shaped handle, a motor disposed within the barrel section, and a battery removably attached to one end of the handle. A trigger may be movably mounted to the housing just above the handle, such that a user can grip the handle with one hand and operate the trigger simultaneously. A control circuit may be disposed within the handle or the barrel section, which monitors actuation of the trigger and—depending on the extent to which the trigger is activated—can selectively energize the motor with current from the battery, to cause the motor to rotate at a desired speed. In other words, the trigger may be pressure sensitive and when a user actuates the trigger, the saw can operate at a variable speed based on the amount of pressure the user applies to the trigger. In some embodiments, the trigger may include a safety mechanism to prevent inadvertent activation of the saw.

Additionally, a collar may extend from the barrel above the trigger. The collar may include features that selectively engage the saw blades described herein and secure the saw blades to the reciprocating surgical saw. In some cases, the collar may be spring-loaded to allow insertion and release of the saw blade quickly and securely.

In some embodiments, the barrel section may include a reciprocating head, which is attached at a first end to the collar and at a second end to the motor. The reciprocating head can translate rotational movement of the motor into oscillating/reciprocating movement of the collar.

Of course, although the aforementioned reciprocating saw is known in the art, any other suitable embodiments of reciprocating saws may alternatively be used with the saw blades described herein without departing from the principles of this disclosure.

As will be described herein, specific configurations of each component of the saw blade (e.g., saw interface portion, offset portion, cutting portion) allow the saw blade to resect bony tissues in a plane that is offset from the axis of reciprocation. In certain circumstances, this may allow a user to more easily remove bony tissues to form prepared bone surfaces in preparation for further processing (e.g., broaching) or simply installing one or more prostheses therein. Thus, the saw blades of this disclosure include several structural features to enhance their functionality, strength, and compatibility with the reciprocating saw. Additionally, in various embodiments, portions of the saw blade that selectively interface with the collar (or other component) of a reciprocating saw may be modifiable in order to use the saw blade, as disclosed herein, with various types of reciprocating saw assemblies.

In various embodiments, the saw blades described herein may be configured to interface with, and be driven by, any suitable reciprocating power source or drive component. For example, the saw blades may be compatible with powered surgical handpieces having electric, pneumatic, hydraulic, or other motors, including battery-powered systems, corded systems, and/or console-driven systems. Additionally, the saw interface portion of the saw blade may be configured to removably engage any suitable coupling structure (e.g., a blade collar, chuck, clamp, detent-based coupling, pinned coupling, keyed coupling, dovetail coupling, spline coupling, or other mechanical interface) and may be modified in geometry to complement corresponding engagement features of a particular reciprocating saw assembly while still positioning the cutting portion to cut in a plane offset from the axis of reciprocation as described herein.

By way of non-limiting example, the saw interface portion may include one or more support ribs, engagement tabs, apertures (e.g., through-holes), recesses or pockets, keys, splines, flats, grooves, detents, and/or other indexing or retention features configured to provide stiffness, alignment, and/or anti-rotation relative to the drive component while maintaining the offset cutting configuration described herein.

The above features (and others) will be discussed herein in the context of specific implant types (e.g., an ankle implant). However, it will be understood that the concepts discussed herein are applicable to any suitable procedure, including for any implant (e.g., shoulder, elbow, knee, wrist, hip, clavicle, etc.) or used anywhere in a human (or other animal). Further, although the embodiments described herein are for use in a surgical setting, the concepts described herein may be adapted for use in other applications according to the principles of this disclosure, including, but not limited to, industrial or consumer settings, where it may be advantageous to provide a saw blade having a cutting plane that is offset from an axis of reciprocation.

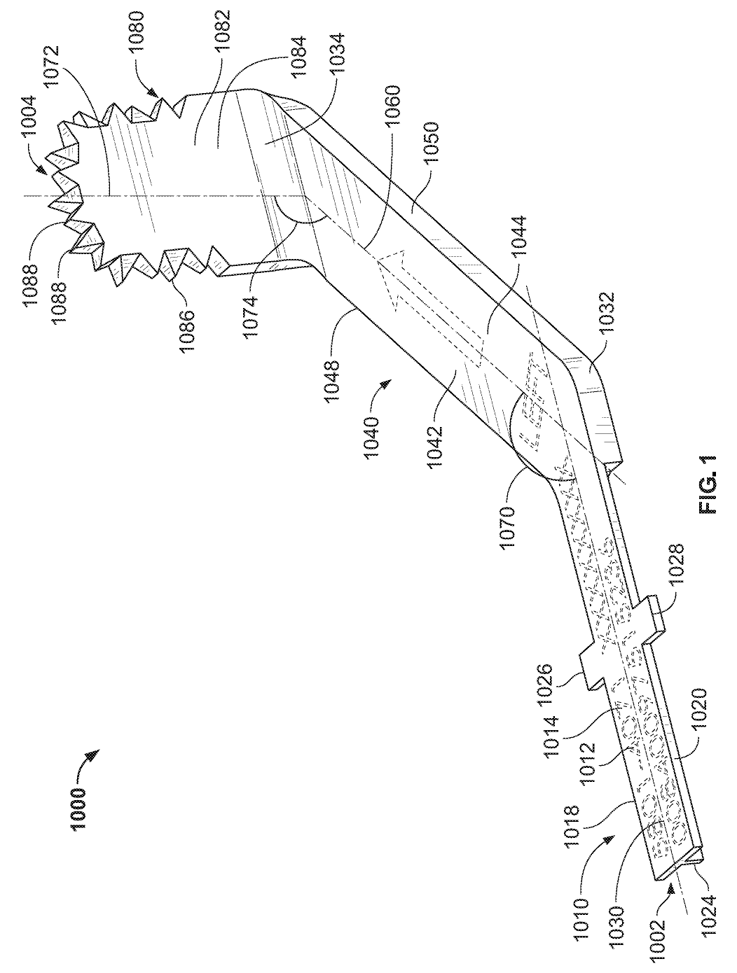

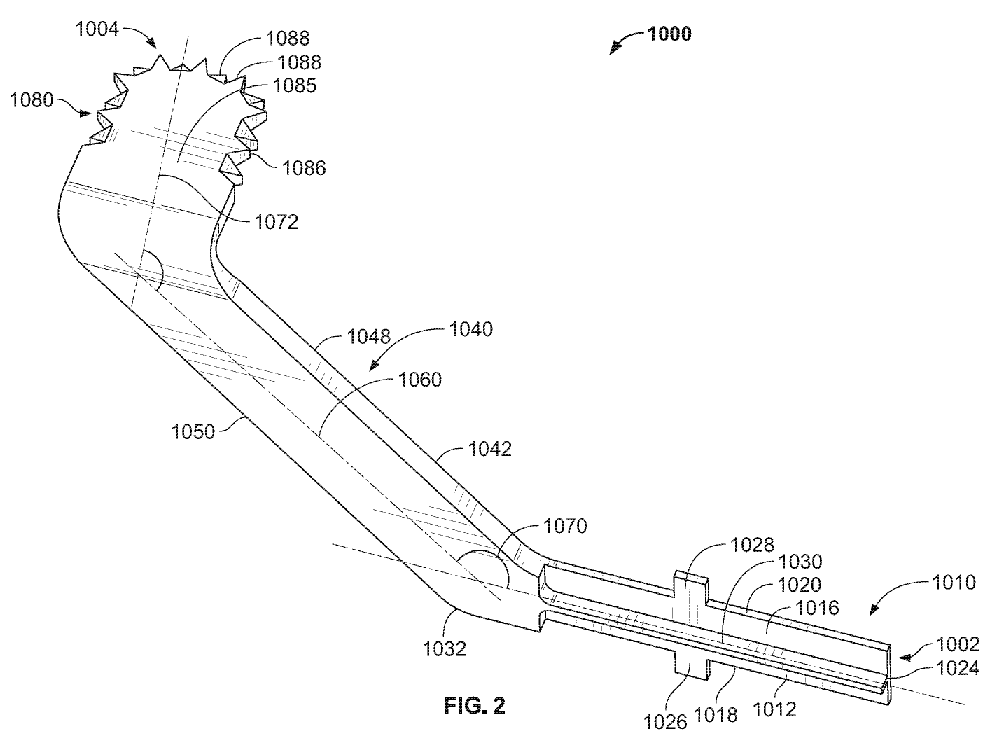

Referring now to FIGS. 1 and 2, one embodiment of an exemplary saw blade 1000 is shown in two perspective views. Generally, the saw blade 1000 may be provided for use with a reciprocating saw (not shown) during the TAR procedure. In this embodiment, the saw blade 1000 includes a first end 1002 and a second end 1004, wherein a saw interface portion 1010 is positioned at the first end 1002 and a cutting portion 1080 is positioned at the second end 1004. The saw blade 1000 includes an offset portion 1040 extending between the saw interface portion 1010 and the cutting portion 1080. The saw blade 1000 may be shaped and oriented such that a cutting or resecting surface of the saw blade 1000 is offset in distance from a surface coupled to the reciprocating saw that is coaxial with an axis of reciprocation. For instance, the cutting surface may be perpendicular to an offset plane, wherein the offset plane is measured relative to the axis of reciprocation. Each feature will be described in detail below.

In some embodiments, the saw interface portion 1010 provides a temporary securement point between the saw blade 1000 (e.g., via one or more retention features) and certain receiving features of the reciprocating saw, such as a blade collar. As used herein, the combination of the reciprocating saw and saw blade 1000 (or any other embodiment of a saw blade) will be referred to as a “reciprocating saw assembly.” In the embodiment shown, the first planar surface 1012 is shown as relatively flat (i.e., for easy insertion into the blade collar). In some embodiments, the saw interface portion 1010 can be aligned along a first axis 1030, wherein the first axis 1030 is parallel or coaxial with the axis of reciprocation of the reciprocating saw (e.g., the linear axis of actuation of a drive component of the reciprocating saw). In some aspects, the saw interface portion 1010 may comprise a generally rectangular or quadrilateral shape, but in other aspects, the saw interface portion 1010 may comprise any suitable shape for selectively engaging the blade collar (e.g., circular or cylindrical, hexagonal, other polygonal shapes).

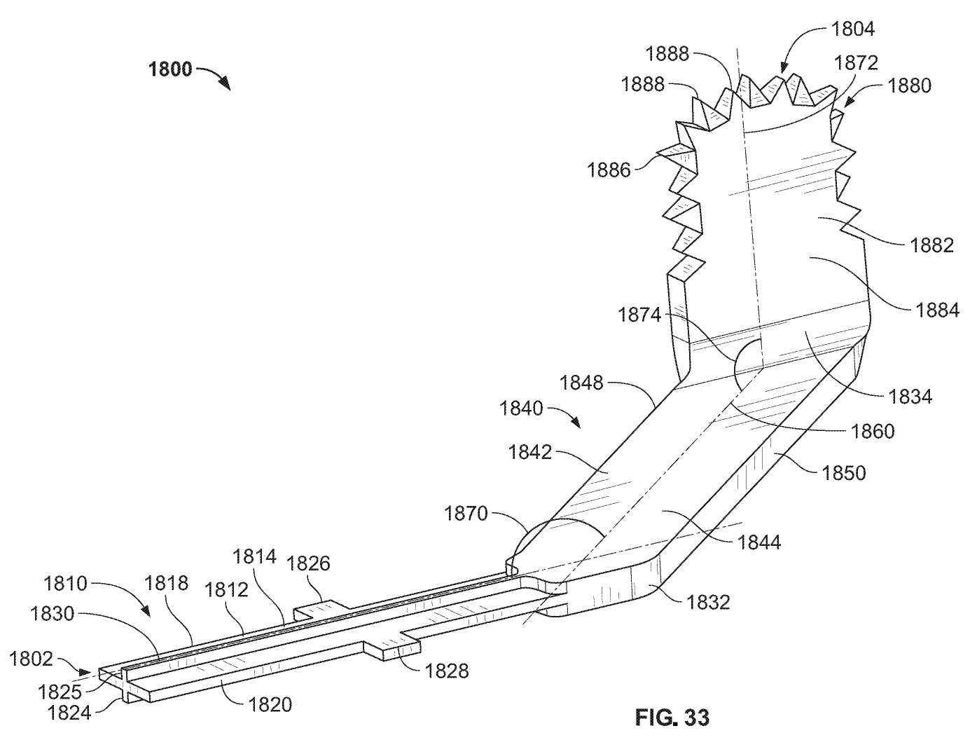

In some embodiments, the first planar surface 1012 comprises a first side 1014 opposite a second side 1016, wherein a retention feature provided in the form of a support rib 1024 may extend lengthwise and/or perpendicularly from the second side 1016. By extending along the length of the saw interface portion 1010, the first planar surface 1012 with the support rib 1024 can form a T-shaped intersection that can be removably received by (or inserted into) the blade collar to form the reciprocating saw assembly. The T-shape formed by the first planar surface 1012 and support rib 1024 provides reinforced stiffness to the first planar surface 1012, which may prevent the first planar surface 1012 from bending under normal forces during use. Additionally, the support rib 1024 may prevent the saw blade 1000 from sliding medial-laterally during use. In alternate embodiments, the saw interface portion 1010 may comprise additional support ribs such that a first support rib extends lengthwise and/or perpendicularly from the first side 1014 and a second support rib extends lengthwise and/or perpendicularly from the second side 1016. In this way, the first planar surface 1012 with the first and the second support ribs can form a cross-shaped or plus-shaped intersection (see, e.g., FIG. 33) that can be removably received by the blade collar. Of course, in some embodiments, additional support ribs may extend from the first planar surface 1012 without departing from the principles of this disclosure.

In some embodiments, the first planar surface 1012 comprises a first edge 1018 and a second edge 1020, wherein the first and second edges 1018, 1020 define lateral boundaries of the saw interface portion 1010. In some cases, a first collar engagement tab 1026 and a second collar engagement tab 1028 extend laterally or outwardly from the first edge 1018 and the second edge 1020, respectively. In some aspects, the first collar engagement tab 1026 and the second collar engagement tab 1028 may provide additional reinforced stiffness to the saw blade 1000 when it is inserted into the blade collar. In other aspects, the first collar engagement tab 1026 and the second collar engagement tab 1028 may prevent the saw blade 1000 from anterior-posterior sliding during use. Although the collar engagement tabs 1026, 1028 are shown as rectangular in shape, they may have any suitable alternative shape without departing from the principles of this disclosure.

In some embodiments, the offset portion 1040 can extend from the saw interface portion 1010 via an elbow bend 1032. The offset portion 1040 bridges/connects the saw interface portion 1010 with the cutting portion 1080. For instance, the elbow bend 1032 may transition the saw interface portion 1010 to the offset portion 1040 via an angle/bend by connecting the first edge 1018 with the first edge 1048 and the second edge 1020 with the second edge 1050. In this way, the offset portion 1040 allows the user to cut or remove bone in otherwise difficult to reach places by offsetting the cutting portion 1080 from the first axis 1030 (which is coaxial with the axis of reciprocation). In some cases, the elbow bend 1032 may comprise smooth or rounded edges, while in other cases, the elbow bend 1032 may comprise sharp or pointed edges.

In some embodiments, the offset portion 1040 can include a second planar surface 1042 having a first side 1044, a second side 1046, a first edge 1048, and a second edge 1050. In the embodiment shown, the second planar surface 1042 is shown as relatively flat and co-planar with the first planar surface 1012. In other embodiments, the second planar surface 1042 may be curved, angled, or otherwise irregularly shaped or oriented based on patient-specific considerations. In certain embodiments, the second planar surface 1042 may not be co-planar with the first planar surface 1012. For instance, the elbow bend 1032 may raise or lower the second planar surface 1042 relative to the first planar surface 1012 to form a sloped or step-wise transition between the surfaces 1012, 1042. In this way, offset portion 1040 may be configured to position the cutting portion 1080 in any suitable position relative to the saw interface portion 1010.

In some embodiments, the offset portion 1040 can be aligned along a second axis 1060, wherein the second axis 1060 is oriented at an offset angle 1070 relative to the first axis 1030. As such, the offset portion 1040 is oriented at the offset angle 1070 relative to the saw interface portion 1010 via the elbow bend 1032. In some cases, the offset angle 1070 can be an obtuse angle while in other cases, the offset angle 1070 can be a right angle or acute angle. For example, the offset angle 1070 can measure between about 10° to 30°, or between about 20° to 40°, or between about 30° to 50°, or between about 40° to 60°, or between about 50° to 70°, or between about 60° to 80°, or between about 70° to 90°, or between about 80° to 100°, or between about 90° to 110°, or between about 100° to 120°, or between about 110° to 130°, or between about 120° to 140°, or between about 130° to 150°, or between about 140° to 160°, or between about 150° to 170°, or between about 160° to 180°, or any other suitable angle. In other cases, the offset portion 1040 can measure between about 90° to 180°.

Additionally, in alternate embodiments where the second planar surface 1042 is not co-planar with the first planar surface 1012, the offset angle 1070 can include both horizontal and vertical components. For instance, the offset portion 1040 may extend non co-planarly from the saw interface portion 1010 such that the second planar surface 1042 and the first planar surface 1012 are not co-planar. In this case, the offset portion 1040 may extend from the saw interface portion 1010 via an offset bend (not shown) oriented at the offset angle 1070, wherein the offset bend raises (or lowers) the second planar surface 1042 relative to the first planar surface 1012 to form a sloped or angular transition between the surfaces 1012, 1042.

In some embodiments, the cutting portion 1080 extends from offset portion 1040. The cutting portion 1080 may comprise a third planar surface 1082 having a first side 1084 and a second side 1085. In the embodiment shown, the cutting portion 1080 includes various features configured to resect bony tissue at a prepared tibial surface. For instance, the cutting portion 1080 further includes at least one edge 1086 having a plurality of cutting teeth 1088 disposed along the at least one edge 1086. The plurality of cutting teeth 1088 may be provided in the form of sharp, pointed protrusions, perforations, or serrations designed to cut through bony tissues by sliding back-and-forth along the prepared tibial surface. In some cases, the plurality of cutting teeth 1088 may be disposed on a portion of the at least one edge 1086 (e.g., a top portion), while in other cases, the plurality of cutting teeth 1088 may be disposed along any suitable edge, end, or surface of the cutting portion 1080.

In some embodiments, the third planar surface 1082 may be aligned along a third axis 1072, wherein the third axis 1072 is oriented at a cutting angle 1074 relative to the second axis 1060. In this way, the cutting portion 1080 is oriented at the cutting angle 1074 relative to the offset portion 1040. In some cases, the cutting angle 1074 can be a right angle while in other cases, the cutting angle 1074 can be an obtuse or acute angle. For example, the cutting angle 1074 can measure between about 0° to 20°, or between about 10° to 30°, or between about 20° to 40°, or between about 30° to 50°, or between about 40° to 60°, or between about 50° to 70°, or between about 60° to 80°, or between about 70° to 90°, or between about 80° to 100°, or between about 90° to 110°, or between about 100° to 120°, or between about 110° to 130°, or between about 120° to 140°, or between about 130° to 150°, or between about 140° to 160°, or between about 150° to 170°, or between about 160° to 180°, or any other suitable angle. In other cases, the cutting angle 1074 can measure between about 0° to 90°.

Additionally, the cutting angle 1074 includes both horizontal and vertical components such that the cutting portion 1080 extends non co-planarly from the offset portion 1040 (i.e., the second planar surface 1042 and the third planar surface 1082 are not co-planar). For instance, the cutting portion 1080 may extend from the offset portion 1040 via a transition bend 1034 oriented at the cutting angle 1074, wherein the transition bend 1034 raises (or lowers) the third planar surface 1082 relative to the second planar surface 1042 to form a sloped or angular transition between the surfaces 1042, 1082. In some cases, the transition bend 1034 may be gradual (e.g., curved, rounded), while in other cases, the transition bend 1034 may be steep.

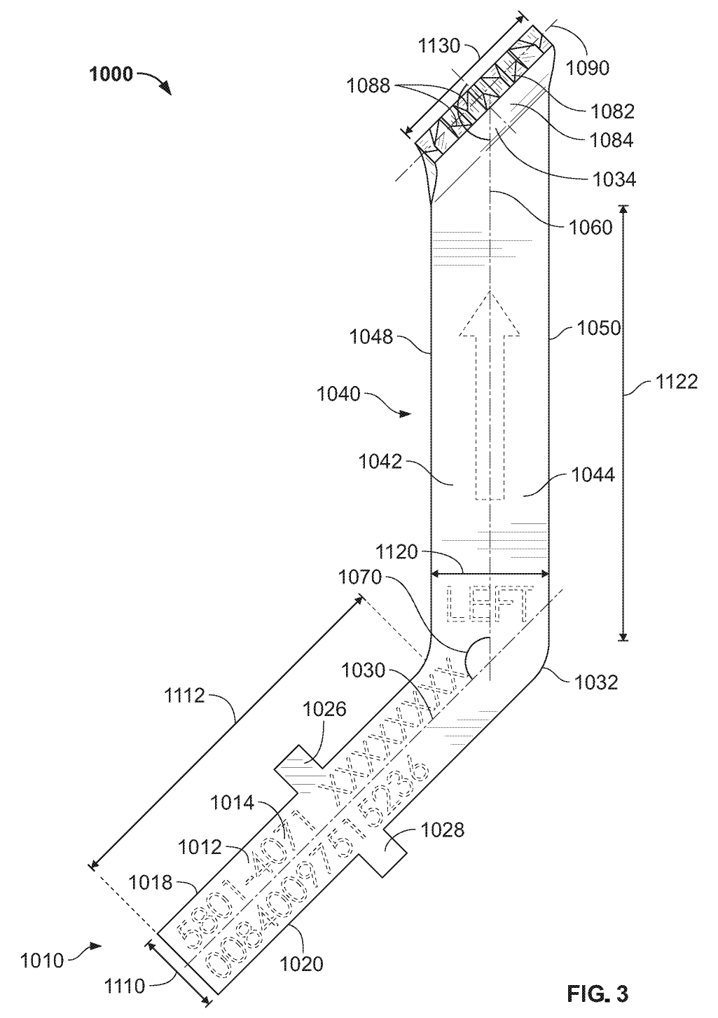

In some embodiments, and with additional reference to FIG. 3, via the combination of the offset angle 1070 and the cutting angle 1074, a cutting axis 1090 may be substantially parallel to the first axis 1030 while oriented a distance away in a different plane. Thus, as the reciprocating saw assembly actuates along the first axis 1030, the plurality of cutting teeth 1088 can resect bony tissues along the cutting axis 1090 (or along a plane parallel to the cutting axis 1090), thereby forming one or more slots into the prepared tibial surface.

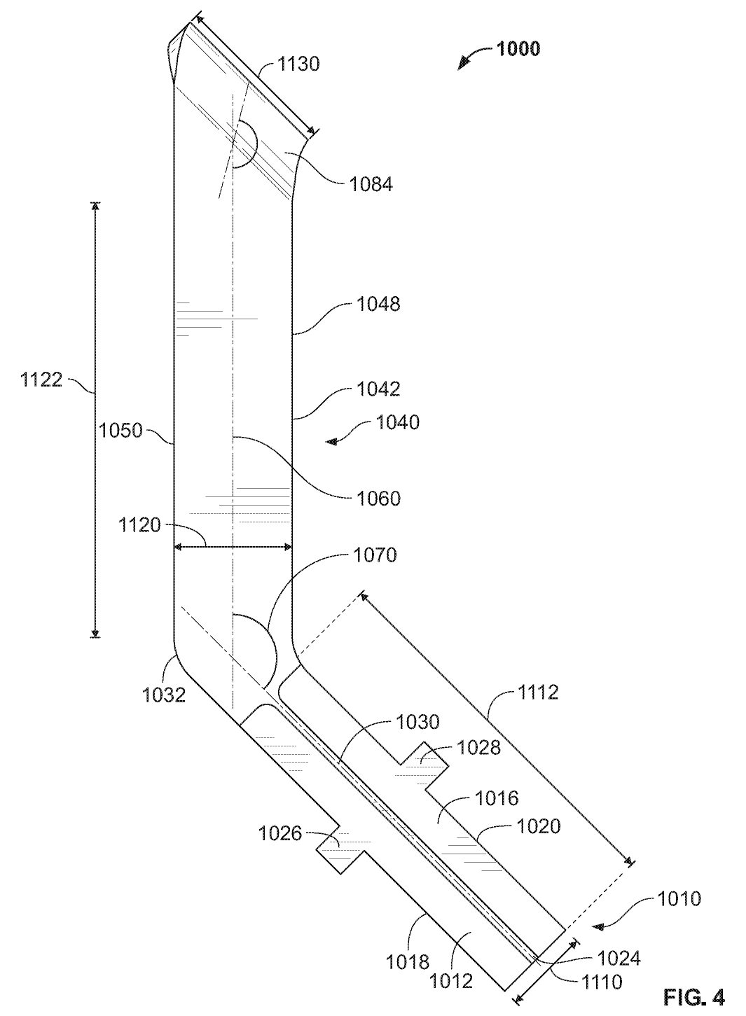

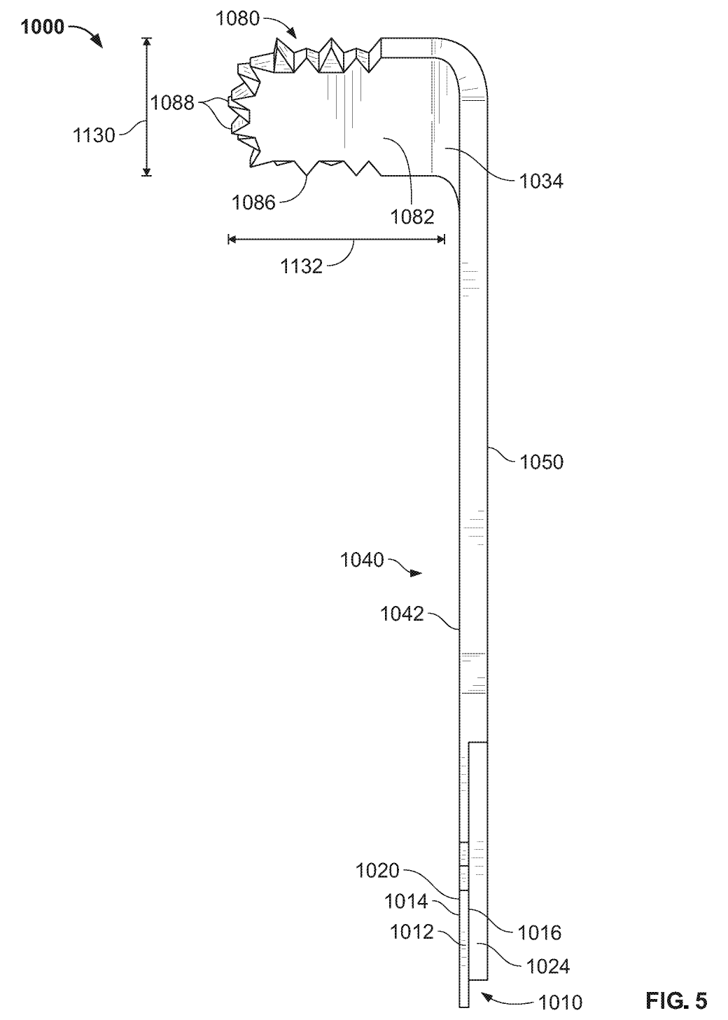

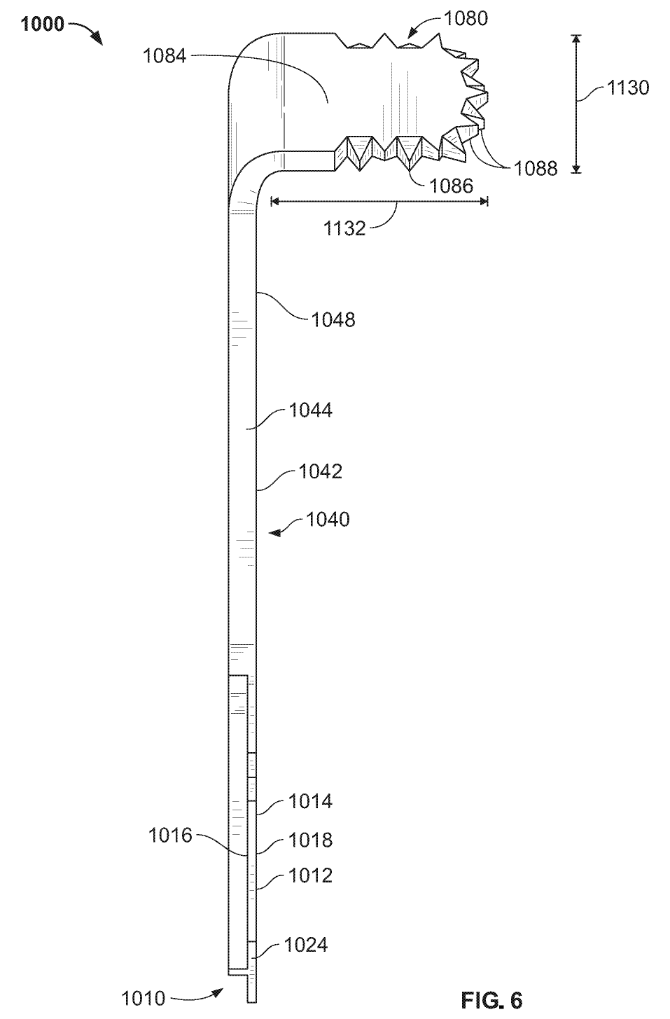

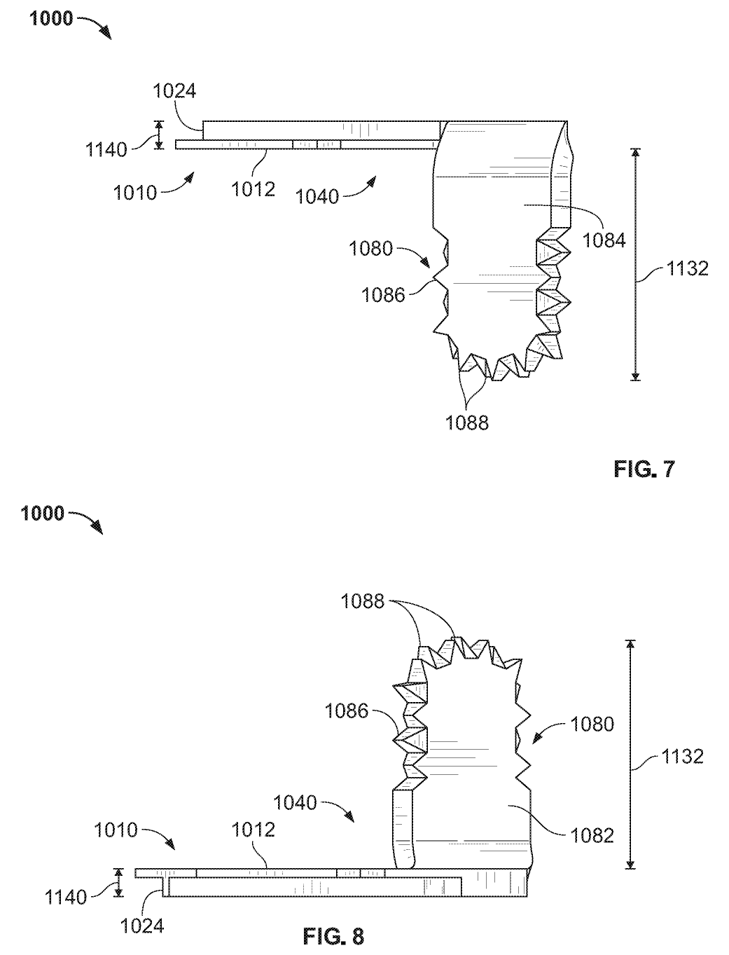

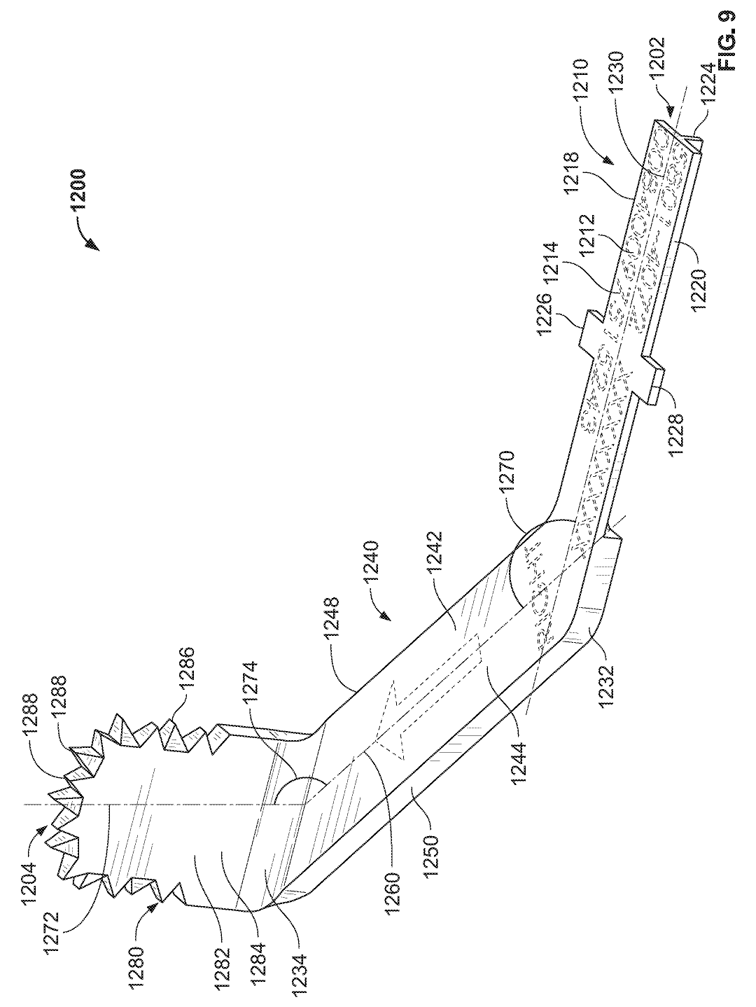

Referring now to FIGS. 3-8, several exemplary and non-limiting dimensions are shown for the saw blade 1000, according to various embodiments.

In one embodiment, the saw interface portion 1010 may include a width 1110 extending from the first edge 1018 to the second edge 1020, and measuring between about 2.0 to 4.0 mm, or between about 3.0 mm to 5.0 mm, or between about 4.0 mm to 6.0 mm, or between about 5.0 mm to 7.0 mm, or between about 6.0 mm to 8.0 mm, or between about 7.0 mm to 9.0 mm, or between about 8.0 mm to 10.0 mm, or between about 9.0 mm to 11.0 mm, or between about 10.0 mm to 12.0 mm, or any other suitable width without departing from the principles of this disclosure.

In one embodiment, the saw interface portion 1010 may include a length 1112 extending from the first end 1002 to about the elbow bend 1032, and measuring between about 15.0 mm to 18.0 mm, or between about 17.5 mm to 20.5 mm, or between about 18.0 mm to 21.0 mm, or between about 19.5 mm to 22.5 mm, or between about 21.0 mm to 24.0 mm, or between about 22.5 mm to 25.5 mm, or between about 24.0 mm to 27.0 mm, or between about 25.5 mm to 28.5 mm, or between about 27.0 mm to 30.0 mm, or between about 28.5 mm to 31.5 mm, between about 30.0 mm to 33.0 mm, or between about 31.5 mm to 34.5 mm, or between about 33.0 mm to 36.0 mm, or between about 34.5 mm to 37.5 mm, or between about 36.0 mm to 39.0 mm, or between about 37.5 mm to 40.5 mm, or between about 39.0 mm to 42.0 mm, or any other suitable length without departing from the principles of this disclosure.

In one embodiment, the offset portion 1040 may include a width 1120 extending from the first edge 1048 to the second edge 1050, and measuring between about 4.0 mm to 6.0 mm, or between about 5.0 mm to 7.0 mm, or between about 6.0 mm to 8.0 mm, or between about 7.0 mm to 9.0 mm, or between about 8.0 mm to 10.0 mm, or between about 9.0 mm to 11.0 mm, or between about 10.0 mm to 12.0 mm, or between about 11.0 mm to 13.0 mm, or between about 12.0 mm to 14.0 mm, or between about 13.0 mm to 15.0 mm, or any other suitable width without departing from the principles of this disclosure.

In one embodiment, the offset portion 1040 may include a length 1122 extending from about the elbow bend 1032 to about the transition bend 1034 between the offset portion 1040 and the cutting portion 1080, and measuring between about 21.0 mm to 24.0 mm, or between about 22.5 mm to 25.5 mm, or between about 24.0 mm to 27.0 mm, or between about 25.5 mm to 28.5 mm, or between about 27.0 mm to 30.0 mm, or between about 28.5 mm to 31.5 mm, between about 30.0 mm to 33.0 mm, or between about 31.5 mm to 34.5 mm, or between about 33.0 mm to 36.0 mm, or between about 34.5 mm to 37.5 mm, or between about 36.0 mm to 39.0 mm, or between about 37.5 mm to 40.5 mm, or between about 39.0 mm to 42.0 mm, or between about 40.5 mm to 43.5 mm, or between about 42.0 mm to 45.0 mm, or between about 43.5 mm to 46.5 mm, or between about 45.0 mm to 48.0 mm, or between about 47.5 mm to 50.5 mm, or between about 49.0 mm to 52.0 mm, or any other suitable length without departing from the principles of this disclosure.

In one embodiment, the cutting portion 1080 may include a width 1130 extending between opposing ends of the at least one edge 1086, and measuring between about 6.0 mm to 8.0 mm, or between about 7.0 mm to 9.0 mm, or between about 8.0 mm to 10.0 mm, or between about 9.0 mm to 11.0 mm, or between about 10.0 mm to 12.0 mm, or between about 11.0 mm to 13.0 mm, or between about 12.0 mm to 14.0 mm, or between about 13.0 mm to 15.0 mm, or between about 12.0 mm to 14.0 mm, or between about 13.0 mm to 15.0 mm, or between about 14.0 mm to 16.0 mm, or between about 15.0 mm to 17.0 mm, or between about 16.0 mm to 18.0 mm, or between about 17.0 mm to 19.0 mm, or between about 18.0 mm to 20.0 mm, or between about 19.0 mm to 21.0 mm, or between about 20.0 mm to 22.0 mm, or any other suitable width without departing from the principles of this disclosure.

In one embodiment, the cutting portion 1080 may include a length 1132 extending from about the transition bend 1034 to the second end 1004, and measuring between about 6.0 mm to 8.0 mm, or between about 7.0 mm to 9.0 mm, or between about 8.0 mm to 10.0 mm, or between about 9.0 mm to 11.0 mm, or between about 10.0 mm to 12.0 mm, or between about 11.0 mm to 13.0 mm, or between about 12.0 mm to 14.0 mm, or between about 13.0 mm to 15.0 mm, or between about 12.0 mm to 14.0 mm, or between about 13.0 mm to 15.0 mm, or between about 14.0 mm to 16.0 mm, or between about 15.0 mm to 17.0 mm, or between about 16.0 mm to 18.0 mm, or between about 17.0 mm to 19.0 mm, or between about 18.0 mm to 20.0 mm, or any other suitable length without departing from the principles of this disclosure.

In one embodiment, the saw blade 1000 may include a varying thickness 1140 along its body from the first end 1002 and the second end 1004, measuring between about 0.1 mm to 0.5 mm, or between about 0.25 mm to 0.75 mm, or between about 0.5 mm to 1.0 mm, or between about 0.75 mm to 1.25 mm, or between about 1.0 mm to 1.5 mm, or between about 1.25 mm to 1.75 mm, or between about 1.5 mm to 2.0 mm, or between about 1.75 mm to 2.25 mm, or between about 2.0 mm to 3.0 mm, or between about 2.5 mm to 3.5 mm, or between about 3.0 mm to 4.0 mm, or between about 3.5 mm to 4.5 mm, or between about 4.0 mm to 5.0 mm, or any other suitable thickness without departing from the principles of this disclosure. In some cases, the thickness 1140 may increase at around the cutting portion 1080 and may decrease at around the saw interface portion 1010.

According to various embodiments, it will be understood that, as described herein, one or more of the saw blades of this disclosure can include any suitable combination, orientation, number, positioning, and sizing of its subcomponents (e.g., saw interface portions, offset portions, cutting portions), among other features and dimensions. For the sake of brevity, the following descriptions of alternate embodiments contemplated herein include differences from the embodiment of FIGS. 1-8.

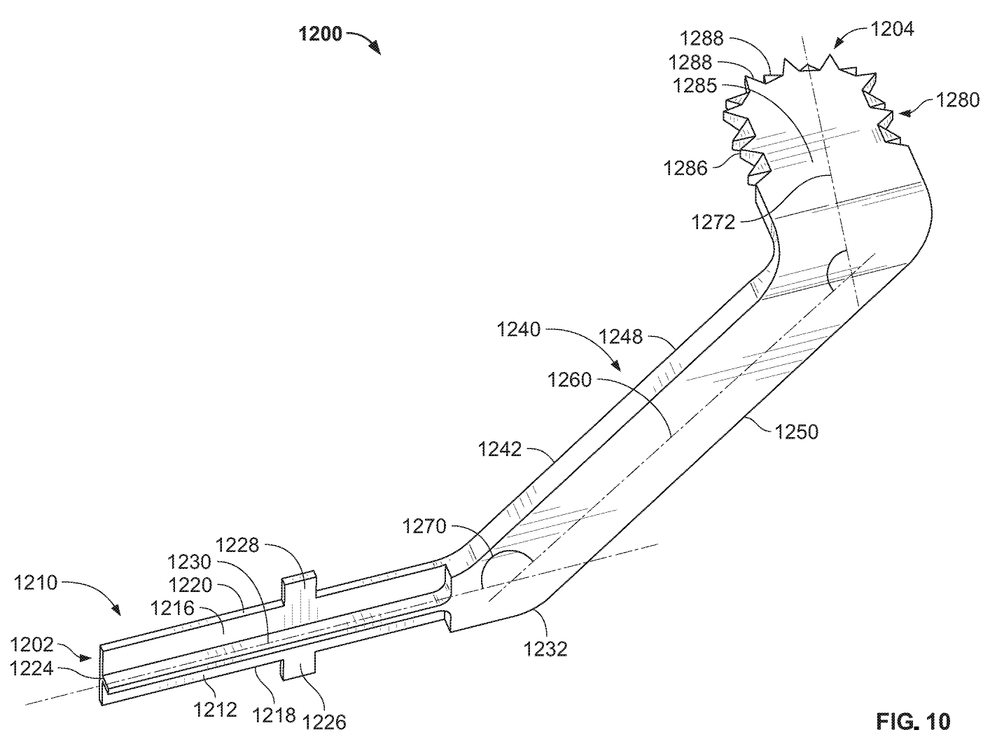

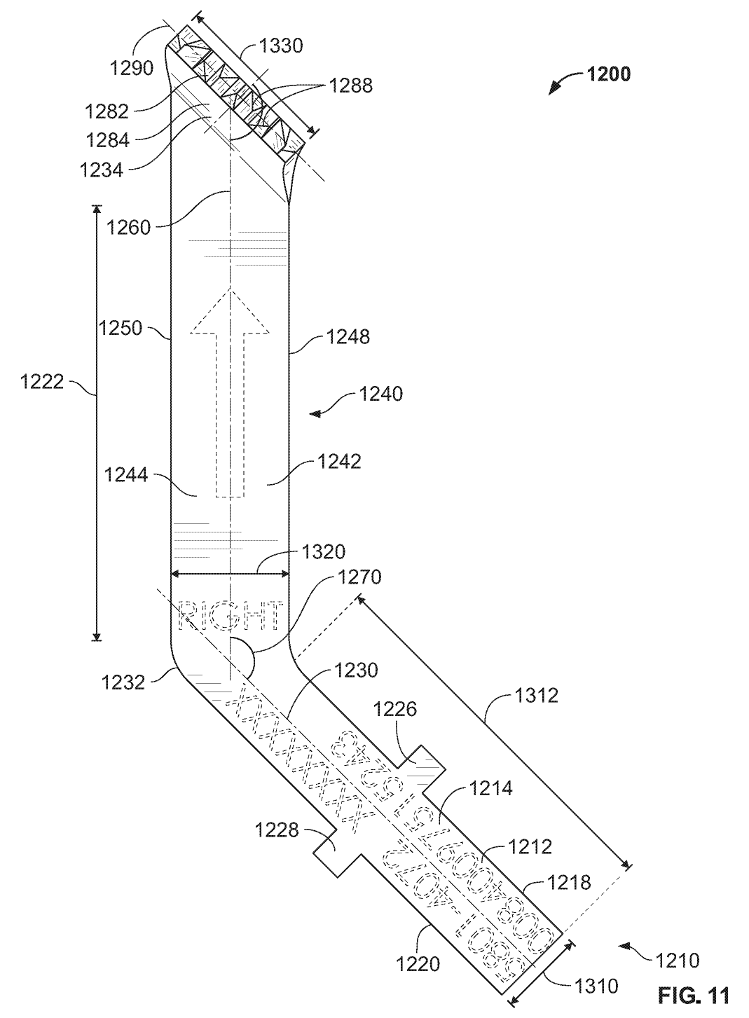

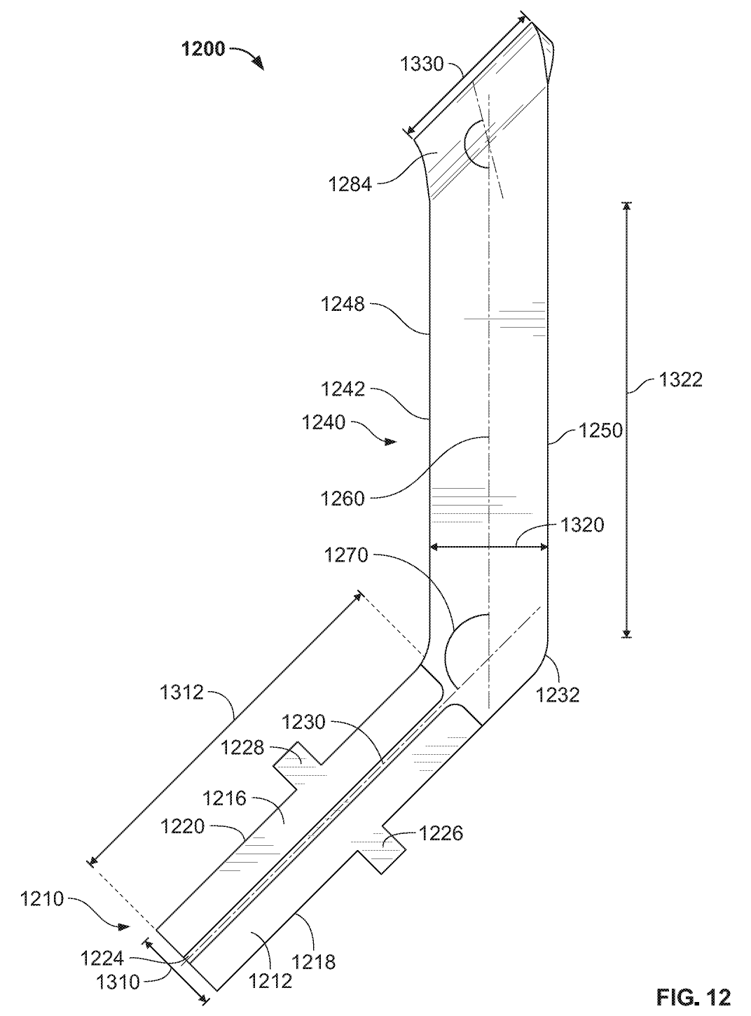

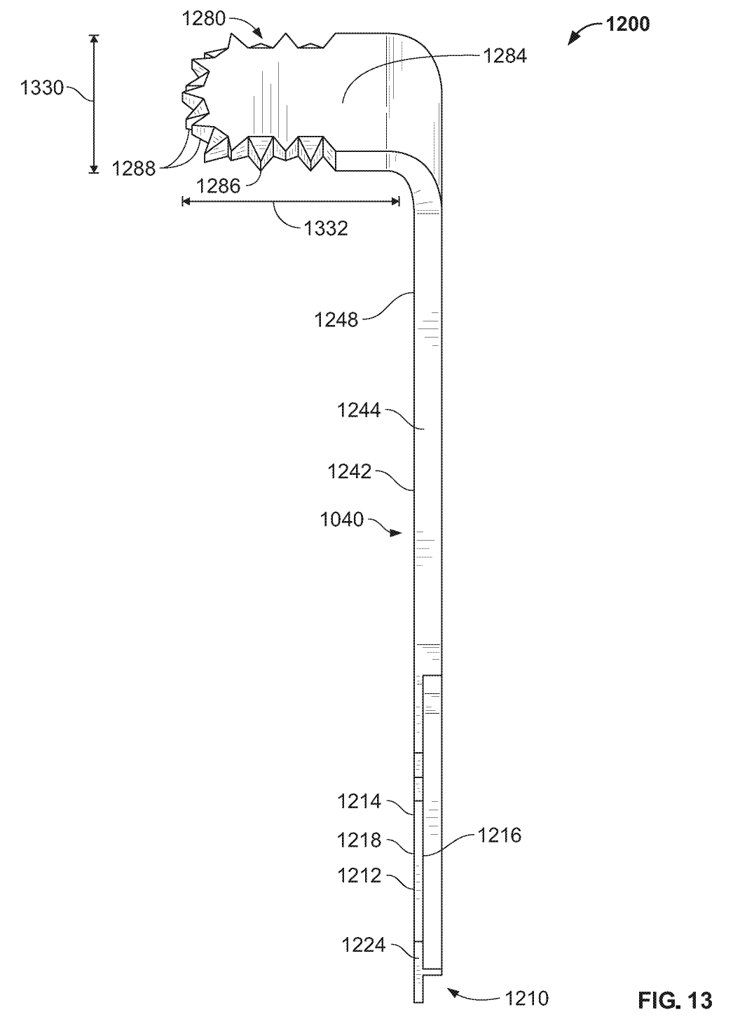

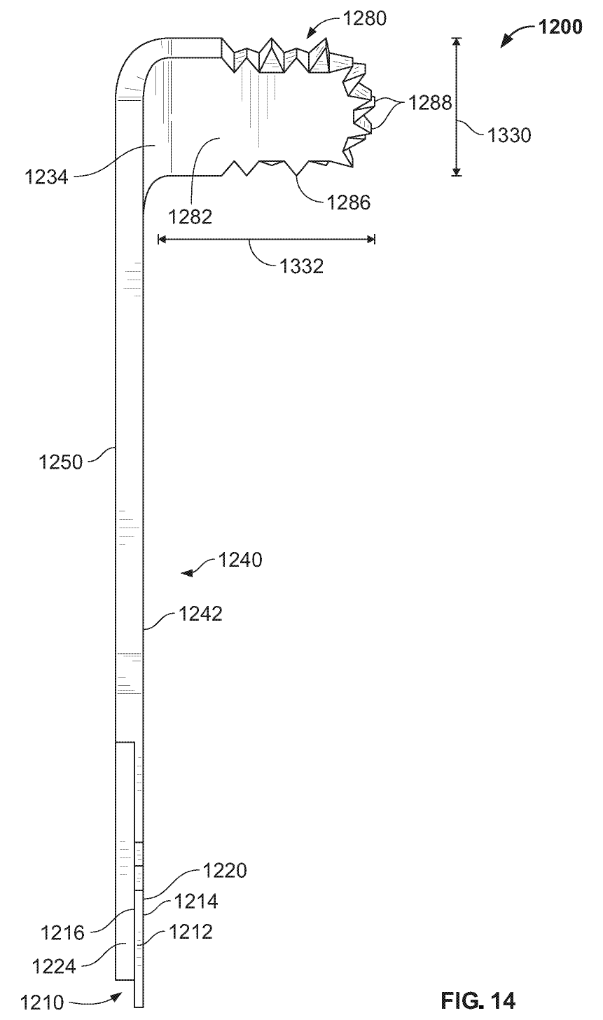

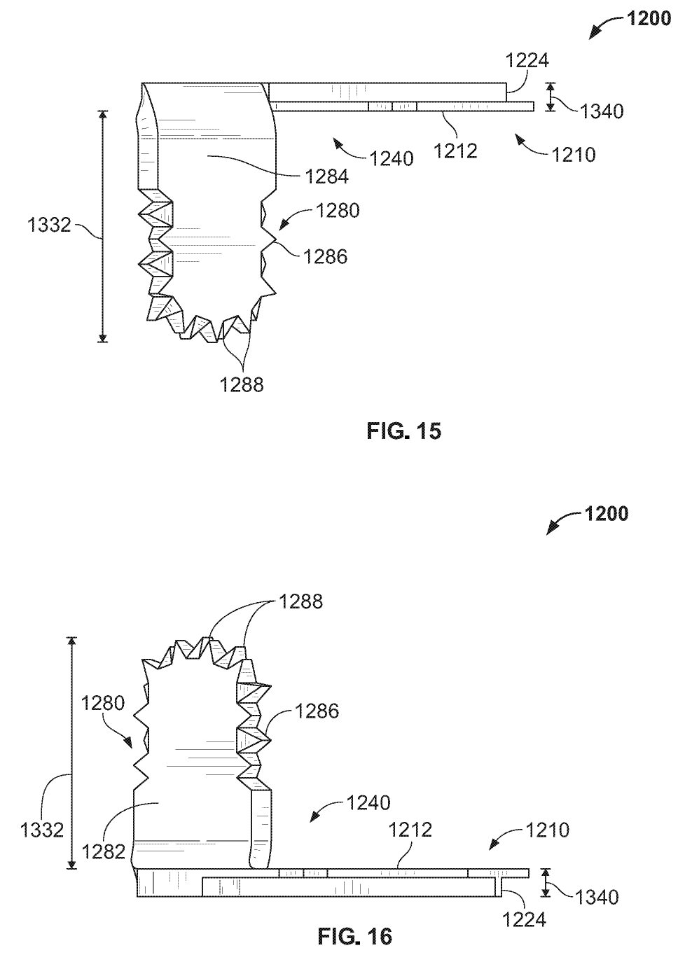

Accordingly, FIGS. 9-16 show another embodiment of an exemplary saw blade 1200 in various views. Similar to the saw blade 1000 of FIGS. 1-8, this embodiment may be provided for use within a reciprocating saw during the TAR procedure. Here, the saw blade 1200 includes a first end 1202 and a second end 1204, wherein a saw interface portion 1210 is positioned at the first end 1202 and a cutting portion 1280 is positioned at the second end 1204. The saw blade 1200 includes an offset portion 1240 extending between the saw interface portion 1210 and the cutting portion 1280. In particular, this embodiment may be configured as a mirror image or complement of saw blade 1000. In one non-limiting example, saw blade 1000 may be oriented to form cuts in a first direction (e.g., “left”) while saw blade 1200 may be oriented to form cuts in a second direction (e.g., “right”). When used in this way, the user can form an X-shaped intramedullary canal into the tibia by using two saw blades, making one cut with saw blade 1000 and a second cut with saw blade 1200. Each feature will be described in detail below.

In some embodiments, the saw interface portion 1210 provides a temporary securement point between the saw blade 1200 (e.g., via one or more retention features) and certain receiving features of the reciprocating saw to form a reciprocating saw assembly. In some embodiments, the saw interface portion 1210 can be aligned along a first axis 1230, wherein the first axis 1230 is coaxial with the axis of reciprocation of the reciprocating saw.

In some embodiments, the first planar surface 1212 comprises a first side 1214 opposite a second side 1216, wherein a retention feature provided in the form of support rib 1224 may extend lengthwise and/or perpendicularly from the second side 1216. The first planar surface 1212 with the support rib 1224 can form a T-shaped intersection that can be removably received by (or inserted into) the blade collar to form the reciprocating saw assembly. In alternate embodiments, the saw interface portion 1210 may comprise any number of additional support ribs, or other comparable structures, and arranged in any suitable orientation.

In some embodiments, the first planar surface 1212 comprises a first edge 1218 and a second edge 1220, wherein the first and second edges 1218, 1220 define lateral boundaries of the saw interface portion 1210. In some cases, a first collar engagement tab 1226 and a second collar engagement tab 1228 extend laterally or outwardly from the first edge 1218 and the second edge 1220, respectively.

In some embodiments, the offset portion 1240 can extend from the saw interface portion 1210 via an elbow bend 1232 to bridge/connect the saw interface portion 1210 with the cutting portion 1280. For instance, the elbow bend 1232 may transition the saw interface portion 1210 to the offset portion 1240 via an angle/bend by connecting the first edge 1218 with the first edge 1248 and the second edge 1220 with the second edge 1250.

In some embodiments, the offset portion 1240 can include a second planar surface 1242 having a first side 1244, a second side 1246, a first edge 1248, and a second edge 1250. In this embodiment, the second planar surface 1242 is shown as co-planar with the first planar surface 1212 while in other embodiments, the second planar surface 1242 may not be co-planar with the first planar surface 1212.

In some embodiments, the offset portion 1240 can be aligned along a second axis 1260, wherein the second axis 1260 is oriented at an offset angle 1270 relative to the first axis 1230. In this way, the offset portion 1240 is oriented at the offset angle 1270 relative to the saw interface portion 1210 via the elbow bend 1232. In some cases, the offset angle 1270 of saw blade 1200 may be complementary to the offset angle 1070 of saw blade 1000.

In some embodiments, the cutting portion 1280 extends from offset portion 1240, the cutting portion 1280 comprising a third planar surface 1282 having a first side 1284 and a second side 1285. Here, the cutting portion 1280 further includes at least one edge 1286 having a plurality of cutting teeth 1288 disposed along the at least one edge 1286. In some cases, the plurality of cutting teeth 1288 may be disposed on a portion of the at least one edge 1286 (e.g., a top portion), while in other cases, the plurality of cutting teeth 1288 may be disposed along any suitable edge, end, or surface.

In some embodiments, the third planar surface 1282 may be aligned along a third axis 1272, wherein the third axis 1272 is oriented at a cutting angle 1274 relative to the second axis 1260. In this way, the cutting portion 1280 is oriented at the cutting angle 1274 relative to the offset portion 1240. In some cases, the cutting angle 1274 of saw blade 1200 may be complementary to the cutting angle 1074 of saw blade 1000. Additionally, in some embodiments, the cutting portion 1280 may extend from the offset portion 1240 via a transition bend 1234 oriented at the cutting angle 1274, wherein the transition bend 1234 raises (or lowers) the third planar surface 1282 relative to the second planar surface 1242 to form a sloped or angular transition between the surfaces 1242, 1282.

In some embodiments, via the combination of the offset angle 1270 and the cutting angle 1274, a cutting axis 1290 may be substantially parallel to the first axis 1230 while oriented a distance away in a different plane. Thus, as the reciprocating saw assembly actuates along the first axis 1230, the plurality of cutting teeth 1288 can resect bony tissues along the cutting axis 1290 (or along a plane parallel to the cutting axis 1290), thereby forming one or more slots into the prepared tibial surface.

According to various embodiments, several exemplary and non-limiting dimensions are shown for the saw blade 1200, which may be substantially similar to the dimensions shown and listed for saw blade 1000.

For instance, in one embodiment, the saw interface portion 1210 may include a width 1310 extending from the first edge 1218 to the second edge 1220. The saw interface portion 1210 may include a length 1312 extending from the first end 1202 to about the elbow bend 1232.

In one embodiment, the offset portion 1240 may include a width 1320 extending from the first edge 1248 to the second edge 1250. The offset portion 1240 may include a length 1322 extending from about the elbow bend 1232 to about the transition bend 1234 between the offset portion 1240 and the cutting portion 1280.

In one embodiment, the cutting portion 1280 may include a width 1330 extending between opposing ends of the at least one edge 1286. The cutting portion 1280 may include a length 1332 extending from about the transition bend 1234 to the second end 1204.

In one embodiment, the saw blade 1200 may include a varying thickness 1340 along its body from the first end 1202 and the second end 1204. In some cases, the thickness 1340 may increase at around the cutting portion 1280 and may decrease at around the saw interface portion 1210.

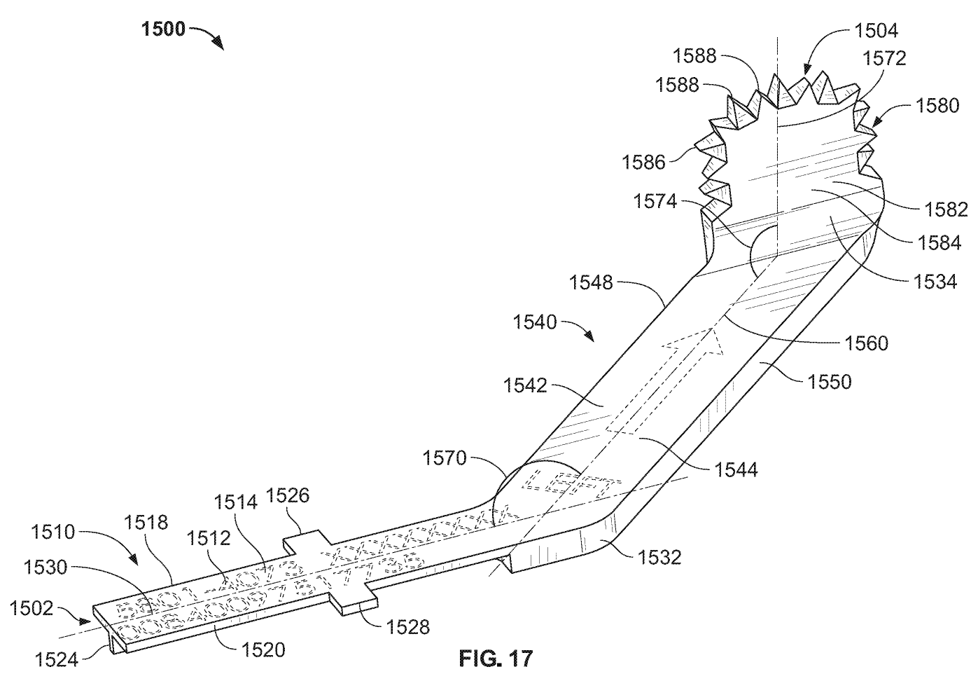

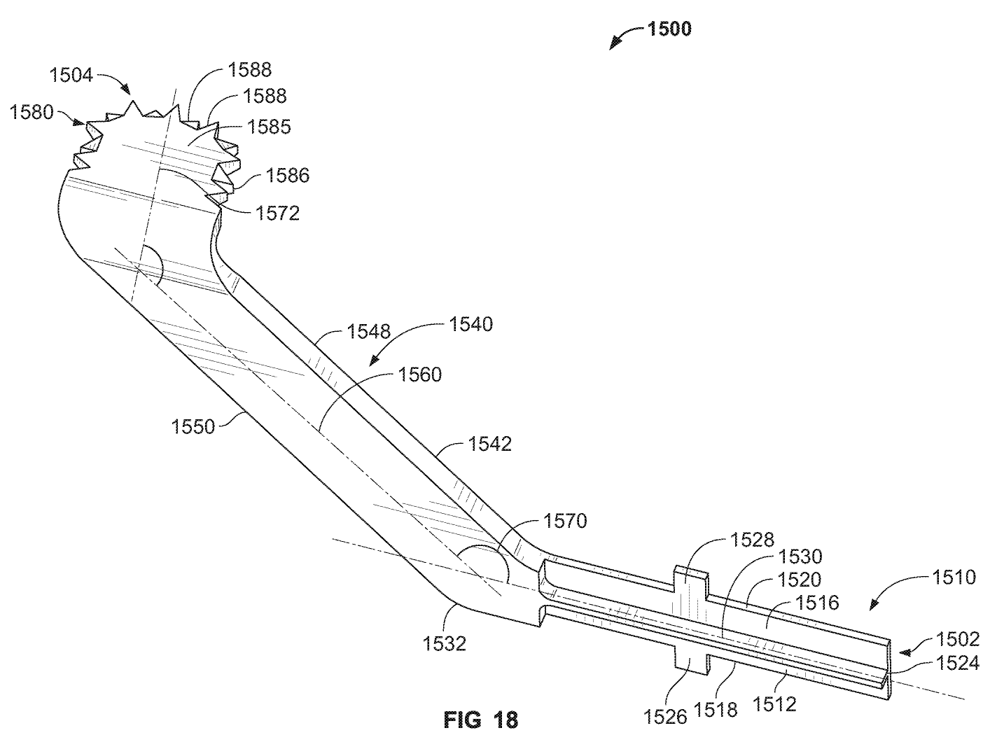

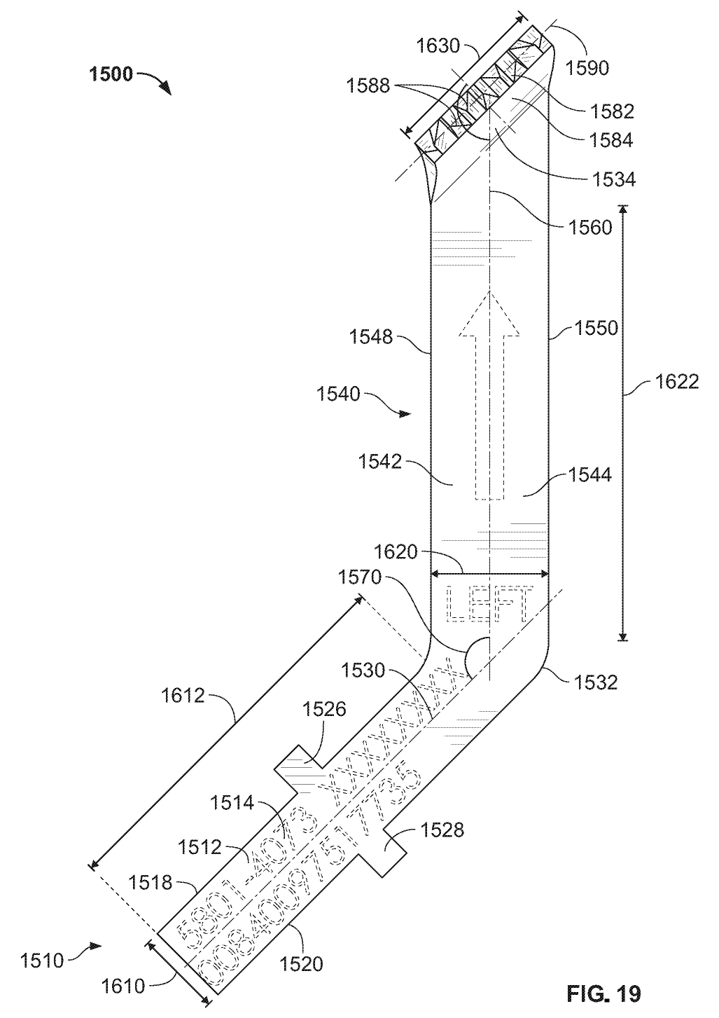

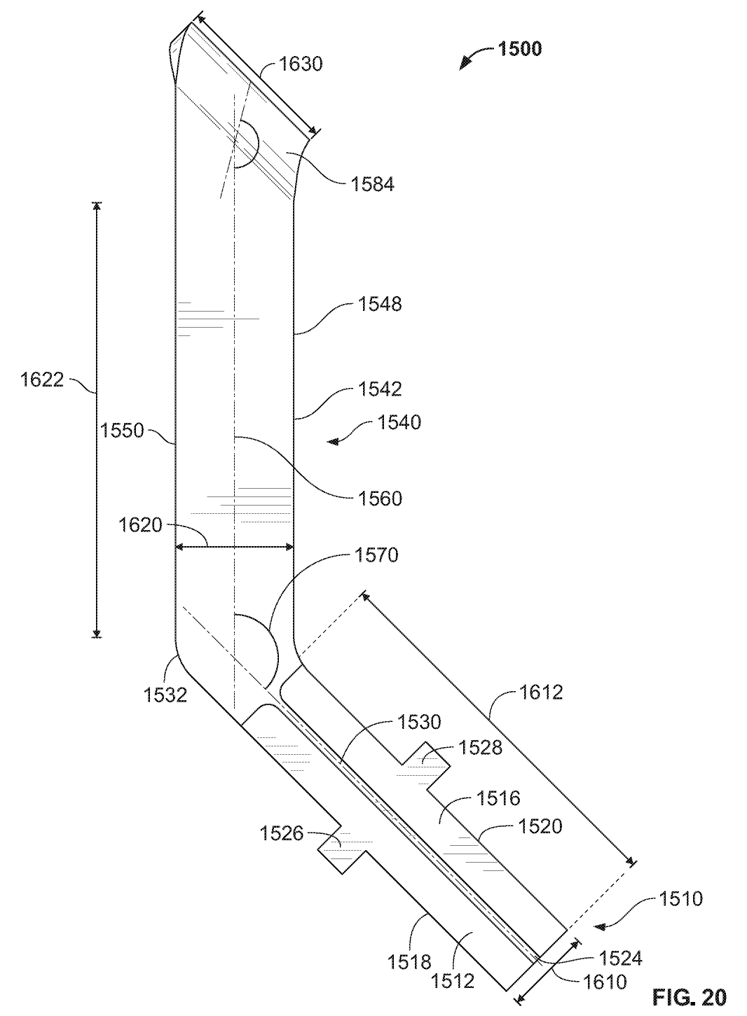

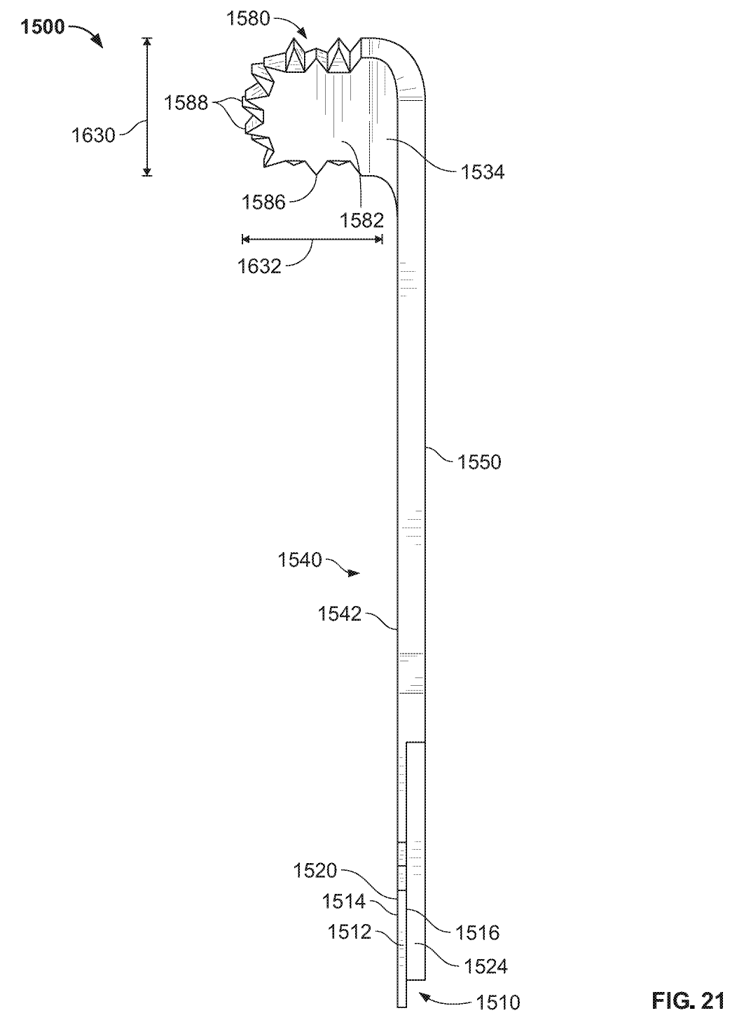

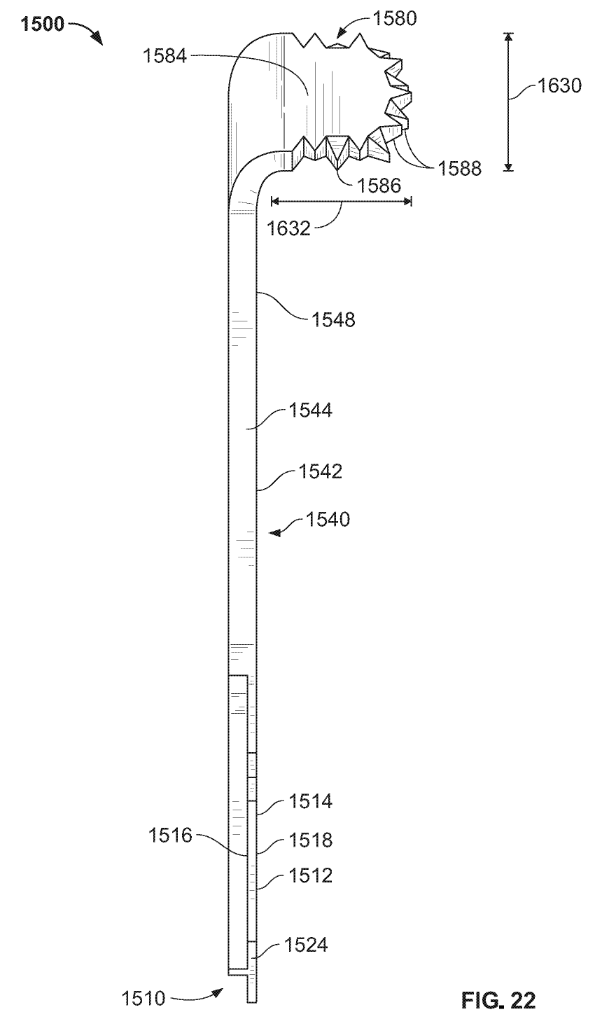

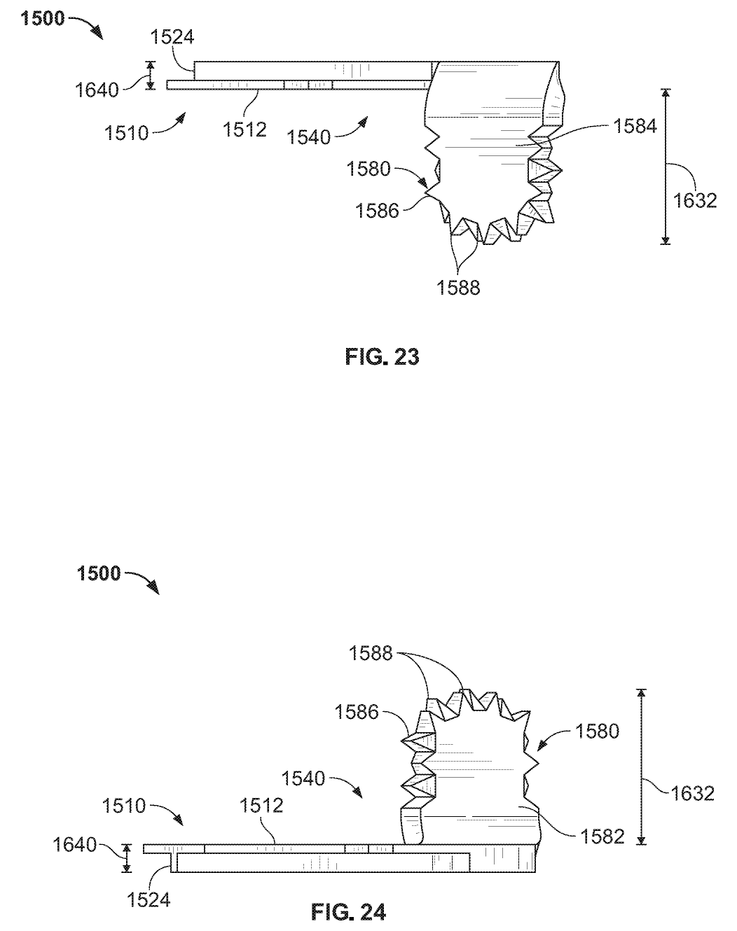

FIGS. 17-24 show another embodiment of an exemplary saw blade 1500 in various views. Similar to the saw blade 1000 of FIGS. 1-8, this embodiment may be provided for use within a reciprocating saw during the TAR procedure. Here, the saw blade 1500 includes a first end 1502 and a second end 1504, wherein a saw interface portion 1510 is positioned at the first end 1502 and a cutting portion 1580 is positioned at the second end 1504. The saw blade 1500 includes an offset portion 1540 extending between the saw interface portion 1510 and the cutting portion 1580. In particular, this embodiment may be configured for smaller patients or for smaller workspaces within the joint. In one non-limiting example, saw blade 1500 may comprise a comparatively smaller and/or differently angled cutting portion 1580 as compared to cutting portion 1080 of saw blade 1000. Each feature will be described in detail below.

In some embodiments, the saw interface portion 1510 provides a temporary securement point between the saw blade 1500 (e.g., via one or more retention features) and certain receiving features of the reciprocating saw to form a reciprocating saw assembly. In some embodiments, the saw interface portion 1510 can be aligned along a first axis 1530, wherein the first axis 1530 is coaxial with the axis of reciprocation of the reciprocating saw.

In some embodiments, the first planar surface 1512 comprises a first side 1514 opposite a second side 1516, wherein a retention feature provided in the form of a support rib 1524 may extend lengthwise and/or perpendicularly from the second side 1516. The first planar surface 1512 with the support rib 1524 can form a T-shaped intersection that can be removably received by (or inserted into) the blade collar to form the reciprocating saw assembly. In alternate embodiments, the saw interface portion 1510 may comprise any number of additional support ribs arranged in any suitable orientation.

In some embodiments, the first planar surface 1512 comprises a first edge 1518 and a second edge 1520, wherein the first and second edges 1518, 1520 define lateral boundaries of the saw interface portion 1510. In some cases, a first collar engagement tab 1526 and a second collar engagement tab 1528 extend laterally or outwardly from the first edge 1518 and the second edge 1520, respectively.

In some embodiments, the offset portion 1540 can extend from the saw interface portion 1510 via an elbow bend 1532 to bridge/connect the saw interface portion 1510 with the cutting portion 1580. For instance, the elbow bend 1532 may transition the saw interface portion 1510 to the offset portion 1540 via an angle/bend by connecting the first edge 1518 with the first edge 1548 and the second edge 1520 with the second edge 1550.

In some embodiments, the offset portion 1540 can include a second planar surface 1542 having a first side 1544, a second side 1546, a first edge 1548, and a second edge 1550. In this embodiment, the second planar surface 1542 is shown as co-planar with the first planar surface 1512 while in other embodiments, the second planar surface 1542 may not be co-planar with the first planar surface 1512.

In some embodiments, the offset portion 1540 can be aligned along a second axis 1560, wherein the second axis 1560 is oriented at an offset angle 1570 relative to the first axis 1530. In this way, the offset portion 1540 is oriented at the offset angle 1570 relative to the saw interface portion 1510 via the elbow bend 1532.

In some embodiments, the cutting portion 1580 extends from offset portion 1540, the cutting portion 1580 comprising a third planar surface 1582 having a first side 1584 and a second side 1585. Here, the cutting portion 1580 further includes at least one edge 1586 having a plurality of cutting teeth 1588 disposed along the at least one edge 1586. In some cases, the plurality of cutting teeth 1588 may be disposed on a portion of the at least one edge 1586 (e.g., a top portion), while in other cases, the plurality of cutting teeth 1588 may be disposed along any suitable edge, end, or surface.

In some embodiments, the third planar surface 1582 may be aligned along a third axis 1572, wherein the third axis 1572 is oriented at a cutting angle 1574 relative to the second axis 1560. In this way, the cutting portion 1580 is oriented at the cutting angle 1574 relative to the offset portion 1540. Additionally, in some embodiments, the cutting portion 1580 may extend from the offset portion 1540 via a transition bend 1534 oriented at the cutting angle 1574, wherein the transition bend 1534 raises (or lowers) the third planar surface 1582 relative to the second planar surface 1542 to form a sloped or angular transition between the surfaces 1542, 1582.

In some embodiments, via the combination of the offset angle 1570 and the cutting angle 1574, a cutting axis 1590 may be substantially parallel to the first axis 1530 while oriented a distance away in a different plane. Thus, as the reciprocating saw assembly actuates along the first axis 1530, the plurality of cutting teeth 1588 can resect bony tissues along the cutting axis 1590 (or along a plane parallel to the cutting axis 1590), thereby forming one or more slots into the prepared tibial surface.

According to various embodiments, several exemplary and non-limiting dimensions are shown for the saw blade 1500, which may be substantially similar to the dimensions shown and listed for saw blade 1000.

For instance, in one embodiment, the saw interface portion 1510 may include a width 1610 extending from the first edge 1518 to the second edge 1520. The saw interface portion 1510 may include a length 1612 extending from the first end 1502 to about the elbow bend 1532.

In one embodiment, the offset portion 1540 may include a width 1620 extending from the first edge 1548 to the second edge 1550. The offset portion 1540 may include a length 1622 extending from about the elbow bend 1532 to about the transition bend 1534 between the offset portion 1540 and the cutting portion 1580.

In one embodiment, the cutting portion 1580 may include a width 1630 extending between opposing ends of the at least one edge 1586. The cutting portion 1580 may include a length 1632 extending from about the transition bend 1534 to the second end 1504.

In one embodiment, the saw blade 1500 may include a varying thickness 1640 along its body from the first end 1502 and the second end 1504. In some cases, the thickness 1640 may increase at around the cutting portion 1580 and may decrease at around the saw interface portion 1510.

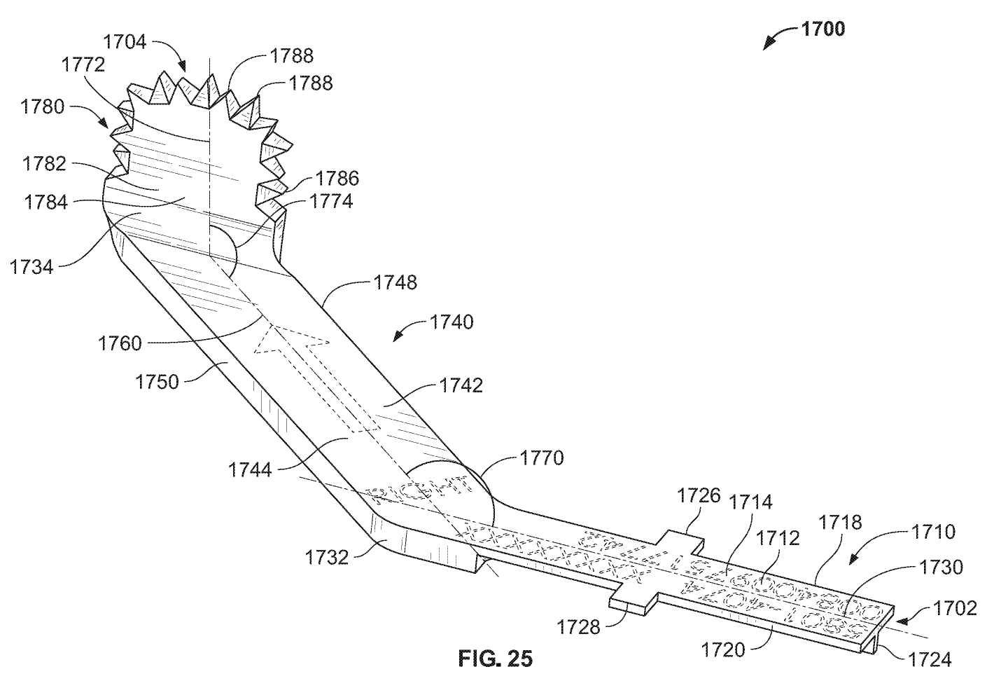

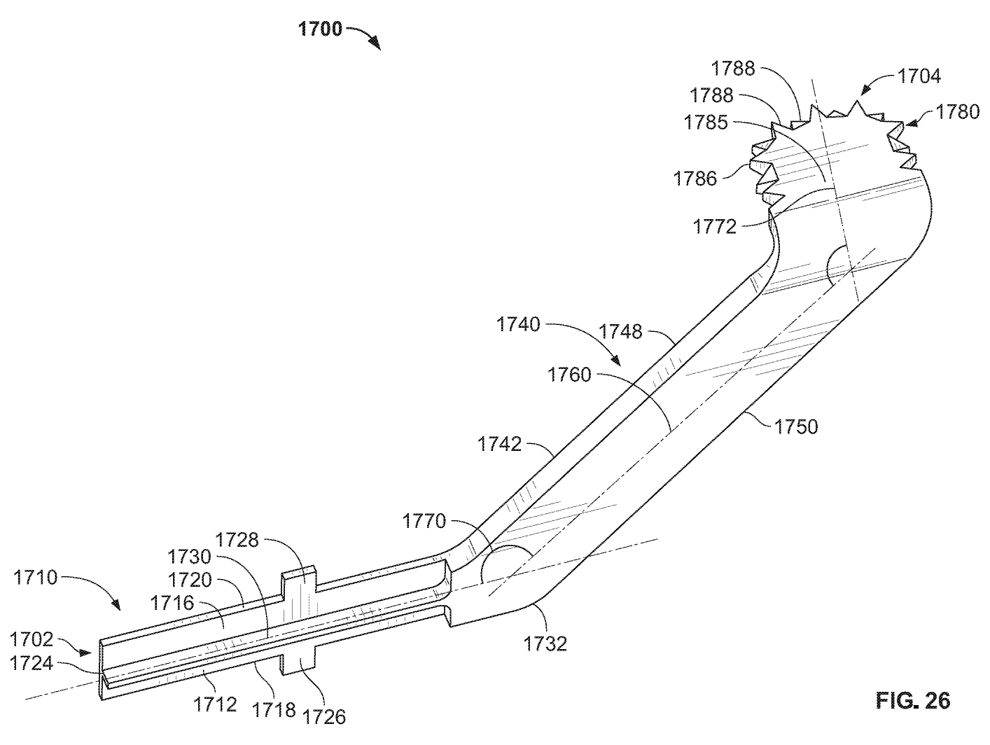

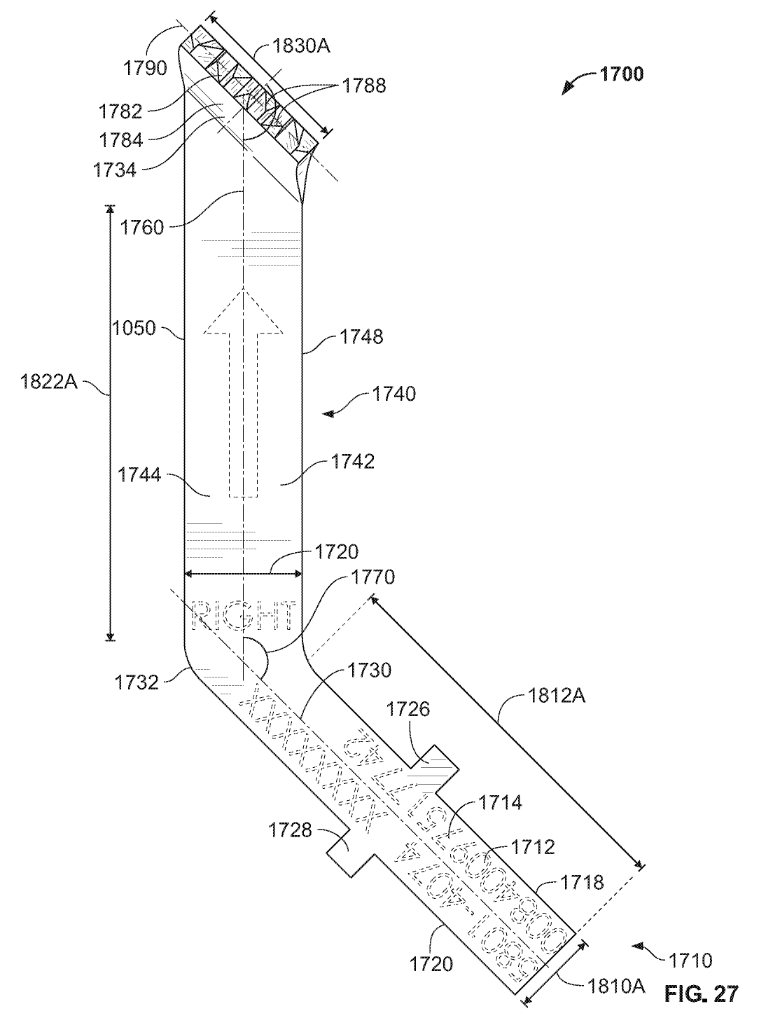

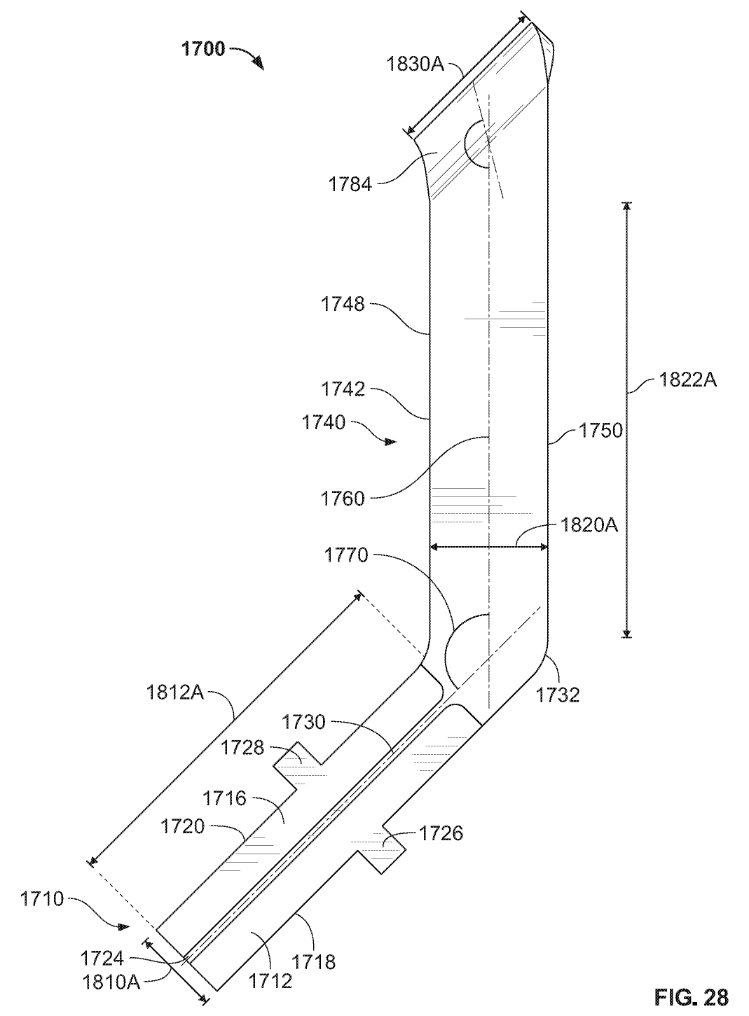

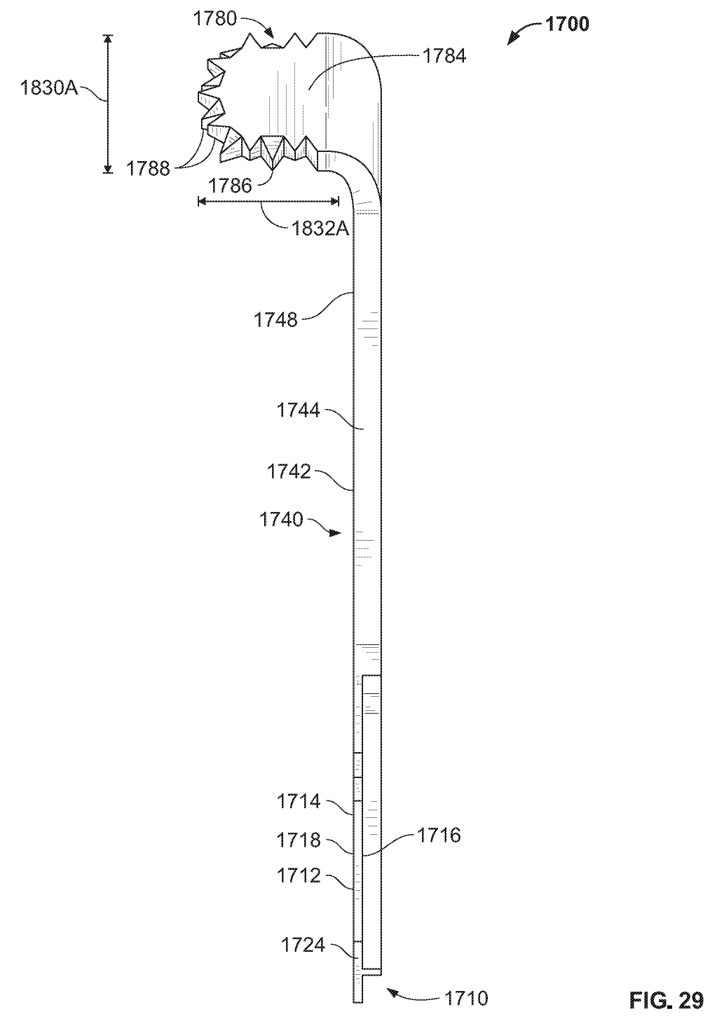

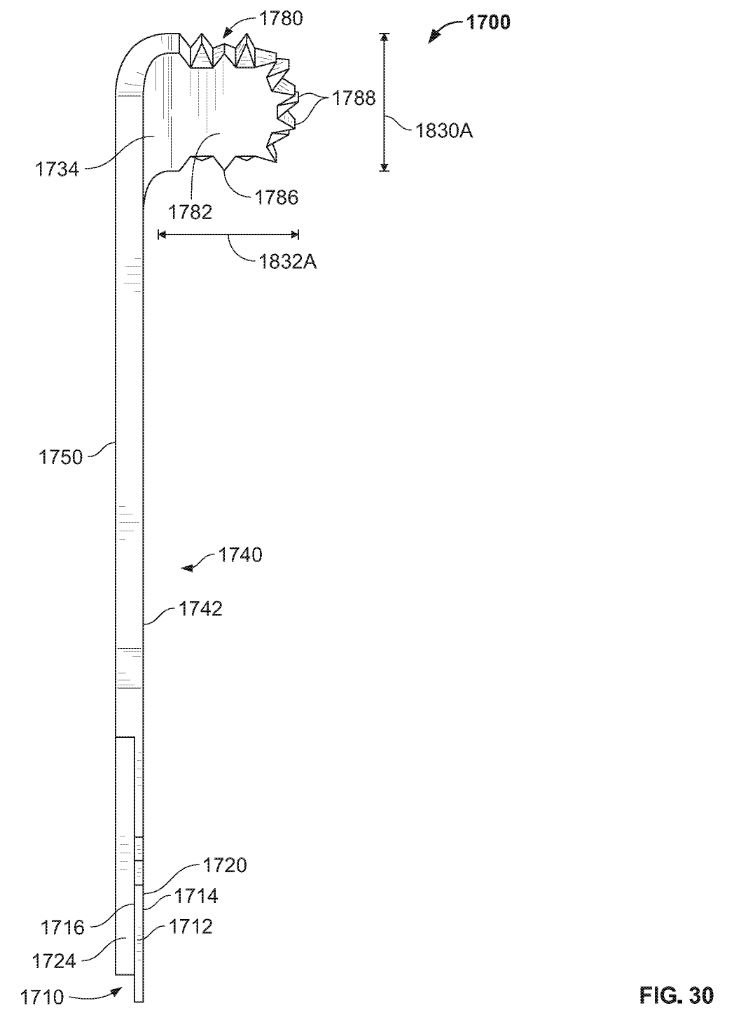

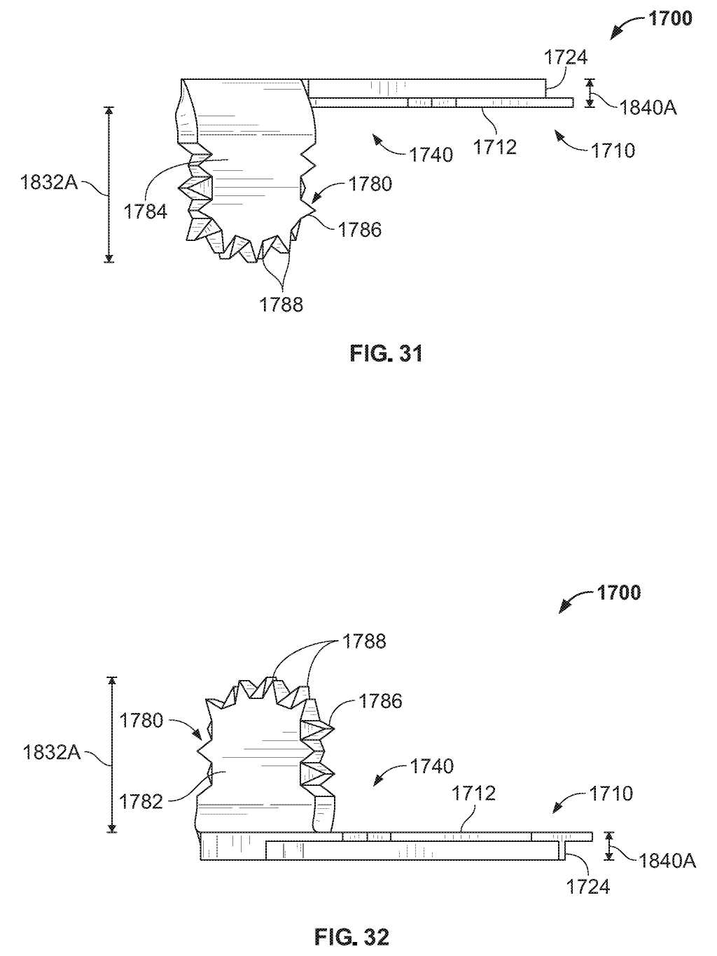

FIGS. 25-32 show another embodiment of an exemplary saw blade 1700 in various views. Similar to the saw blade 1000 of FIGS. 1-8, this embodiment may be provided for use within a reciprocating saw during the TAR procedure. Further, similar to the embodiment of FIGS. 17-24, this embodiment may be configured for smaller patients or for smaller workspaces within the joint. Here, the saw blade 1700 includes a first end 1702 and a second end 1704, wherein a saw interface portion 1710 is positioned at the first end 1702 and a cutting portion 1780 is positioned at the second end 1704. The saw blade 1700 includes an offset portion 1740 extending between the saw interface portion 1710 and the cutting portion 1780. In particular, this embodiment may be configured as a mirror image or complement of saw blade 1500. In one non-limiting example, saw blade 1500 may be oriented to form cuts in a first direction (e.g., “left”) while saw blade 1700 may be oriented to form cuts in a second direction (e.g., “right”). Each feature will be described in detail below.

In some embodiments, the saw interface portion 1710 provides a temporary securement point between the saw blade 1700 (e.g., via one or more retention features) and certain receiving features of the reciprocating saw to form a reciprocating saw assembly. In some embodiments, the saw interface portion 1710 can be aligned along a first axis 1730, wherein the first axis 1730 is coaxial with the axis of reciprocation of the reciprocating saw.

In some embodiments, the first planar surface 1712 comprises a first side 1714 opposite a second side 1716, wherein a retention feature provided in the form of a support rib 1724 may extend lengthwise and/or perpendicularly from the second side 1716. The first planar surface 1712 with the support rib 1724 can form a T-shaped intersection that can be removably received by (or inserted into) the blade collar to form the reciprocating saw assembly. In alternate embodiments, the saw interface portion 1710 may comprise any number of additional support ribs arranged in any suitable orientation.

In some embodiments, the first planar surface 1712 comprises a first edge 1718 and a second edge 1720, wherein the first and second edges 1718, 1720 define lateral boundaries of the saw interface portion 1710. In some cases, a first collar engagement tab 1726 and a second collar engagement tab 1728 extend laterally or outwardly from the first edge 1718 and the second edge 1720, respectively.

In some embodiments, the offset portion 1740 can extend from the saw interface portion 1710 via an elbow bend 1732 to bridge/connect the saw interface portion 1710 with the cutting portion 1780. For instance, the elbow bend 1732 may transition the saw interface portion 1710 to the offset portion 1740 via an angle/bend by connecting the first edge 1718 with the first edge 1748 and the second edge 1720 with the second edge 1750.

In some embodiments, the offset portion 1740 can include a second planar surface 1742 having a first side 1744, a second side 1746, a first edge 1748, and a second edge 1750. In this embodiment, the second planar surface 1742 is shown as co-planar with the first planar surface 1712 while in other embodiments, the second planar surface 1742 may not be co-planar with the first planar surface 1712.

In some embodiments, the offset portion 1740 can be aligned along a second axis 1760, wherein the second axis 1760 is oriented at an offset angle 1770 relative to the first axis 1730. In this way, the offset portion 1740 is oriented at the offset angle 1770 relative to the saw interface portion 1710 via the elbow bend 1732. In some cases, the offset angle 1770 of saw blade 1700 may be complementary to the offset angle 1570 of saw blade 1500.

In some embodiments, the cutting portion 1780 extends from offset portion 1740, the cutting portion 1780 comprising a third planar surface 1782 having a first side 1784 and a second side 1785. Here, the cutting portion 1780 further includes at least one edge 1786 having a plurality of cutting teeth 1788 disposed along the at least one edge 1786. In some cases, the plurality of cutting teeth 1788 may be disposed on a portion of the at least one edge 1786 (e.g., a top portion), while in other cases, the plurality of cutting teeth 1788 may be disposed along any suitable edge, end, or surface.

In some embodiments, the third planar surface 1782 may be aligned along a third axis 1772, wherein the third axis 1772 is oriented at a cutting angle 1774 relative to the second axis 1760. In this way, the cutting portion 1780 is oriented at the cutting angle 1774 relative to the offset portion 1740. In some cases, the cutting angle 1774 of saw blade 1700 may be complementary to the cutting angle 1574 of saw blade 1500. Additionally, in some embodiments, the cutting portion 1780 may extend from the offset portion 1740 via a transition bend 1734 oriented at the cutting angle 1774, wherein the transition bend 1734 raises (or lowers) the third planar surface 1782 relative to the second planar surface 1742 to form a sloped or angular transition between the surfaces 1742, 1782.

In some embodiments, via the combination of the offset angle 1770 and the cutting angle 1774, a cutting axis 1790 may be substantially parallel to the first axis 1730 while oriented a distance away in a different plane. Thus, as the reciprocating saw assembly actuates along the first axis 1730, the plurality of cutting teeth 1788 can resect bony tissues along the cutting axis 1790 (or along a plane parallel to the cutting axis 1790), thereby forming one or more slots into the prepared tibial surface.

According to various embodiments, several exemplary and non-limiting dimensions are shown for the saw blade 1700, which may be substantially similar to the dimensions shown and listed for saw blade 1000.

For instance, in one embodiment, the saw interface portion 1710 may include a width 1810A extending from the first edge 1718 to the second edge 1720. The saw interface portion 1710 may include a length 1812A extending from the first end 1702 to about the elbow bend 1732.

In one embodiment, the offset portion 1740 may include a width 1820A extending from the first edge 1748 to the second edge 1750. The offset portion 1740 may include a length 1822A extending from about the elbow bend 1732 to about the transition bend 1734 between the offset portion 1740 and the cutting portion 1780.

In one embodiment, the cutting portion 1780 may include a width 1830A extending between opposing ends of the at least one edge 1786. The cutting portion 1780 may include a length 1832A extending from about the transition bend 1734 to the second end 1704.

In one embodiment, the saw blade 1700 may include a varying thickness 1840A along its body from the first end 1702 and the second end 1704. In some cases, the thickness 1840A may increase at around the cutting portion 1780 and may decrease at around the saw interface portion 1710.

FIG. 33 shows another embodiment of an exemplary saw blade 1800 in perspective view. Similar to the saw blade 1000 of FIGS. 1-8 and saw blade 1500 of FIGS. 17-24, this embodiment may be provided for use within a reciprocating saw during the TAR procedure. Here, the saw blade 1800 includes a first end 1802 and a second end 1804, wherein a saw interface portion 1810 is positioned at the first end 1802 and a cutting portion 1880 is positioned at the second end 1804. The saw blade 1800 includes an offset portion 1840 extending between the saw interface portion 1810 and the cutting portion 1880. In particular, this embodiment may be configured to provide an enhanced mechanical interface with a drive component of the reciprocating surgical saw and/or a powered blade collar coupled to a power source (e.g., a battery-powered motor). Each feature will be described in detail below.

In some embodiments, the saw interface portion 1810 provides a temporary securement point between the saw blade 1800 (e.g., via one or more retention features) and certain receiving features of the reciprocating saw to form a reciprocating saw assembly. In some embodiments, the saw interface portion 1810 can be aligned along a first axis 1830, wherein the first axis 1830 is coaxial with the axis of reciprocation of the reciprocating saw.

In some embodiments, the first planar surface 1812 comprises a first side 1814 opposite a second side 1816, wherein a retention feature provided in the form of a support rib 1824 may extend lengthwise and/or perpendicularly from the second side 1816. In some embodiments, the retention feature of the saw interface portion 1810 further includes a second support rib 1825 extending lengthwise and/or perpendicularly from the first side 1814, such that the first planar surface 1812 with the support ribs 1824, 1825 forms a plus-shaped profile or plus-shaped intersection that can be removably received by (or inserted into) the blade collar to form the reciprocating saw assembly. As compared to saw blade 1000, the plus-shaped interface may provide greater anti-rotation and reduced looseness within the blade collar during high-load cutting. In this way, the saw interface portion 1810 may provide increased torsional rigidity and bending stiffness, improved resistance to medial-lateral and anterior-posterior play within the blade collar during reciprocation, and more consistent transfer of reciprocating forces from the drive component to the cutting portion 1880. In alternate embodiments, the saw interface portion 1810 may comprise any number of additional support ribs arranged in any suitable orientation.

In some embodiments, the first planar surface 1812 comprises a first edge 1818 and a second edge 1820, wherein the first and second edges 1818, 1820 define lateral boundaries of the saw interface portion 1810. In some cases, a first collar engagement tab 1826 and a second collar engagement tab 1828 extend laterally or outwardly from the first edge 1818 and the second edge 1820, respectively.

In some embodiments, the offset portion 1840 can extend from the saw interface portion 1810 via an elbow bend 1832 to bridge/connect the saw interface portion 1810 with the cutting portion 1880. For instance, the elbow bend 1832 may transition the saw interface portion 1810 to the offset portion 1840 via an angle/bend by connecting the first edge 1818 with the first edge 1848 and the second edge 1820 with the second edge 1850.

In some embodiments, the offset portion 1840 can include a second planar surface 1842 having a first side 1844, a second side 1846, a first edge 1848, and a second edge 1850. In this embodiment, the second planar surface 1842 is shown as co-planar with the first planar surface 1812 while in other embodiments, the second planar surface 1842 may not be co-planar with the first planar surface 1812.

In some embodiments, the offset portion 1840 can be aligned along a second axis 1860, wherein the second axis 1860 is oriented at an offset angle 1870 relative to the first axis 1830. In this way, the offset portion 1840 is oriented at the offset angle 1870 relative to the saw interface portion 1810 via the elbow bend 1832.

In some embodiments, the cutting portion 1880 extends from offset portion 1840, the cutting portion 1880 comprising a third planar surface 1882 having a first side 1884 and a second side 1885. Here, the cutting portion 1880 further includes at least one edge 1886 having a plurality of cutting teeth 1888 disposed along the at least one edge 1886. In some cases, the plurality of cutting teeth 1888 may be disposed on a portion of the at least one edge 1886 (e.g., a top portion), while in other cases, the plurality of cutting teeth 1888 may be disposed along any suitable edge, end, or surface.

In some embodiments, the third planar surface 1882 may be aligned along a third axis 1872, wherein the third axis 1872 is oriented at a cutting angle 1874 relative to the second axis 1860. In this way, the cutting portion 1880 is oriented at the cutting angle 1874 relative to the offset portion 1840. Additionally, in some embodiments, the cutting portion 1880 may extend from the offset portion 1840 via a transition bend 1834 oriented at the cutting angle 1874, wherein the transition bend 1834 raises (or lowers) the third planar surface 1882 relative to the second planar surface 1842 to form a sloped or angular transition between the surfaces 1842, 1882.

In some embodiments, via the combination of the offset angle 1870 and the cutting angle 1874, a cutting axis 1890 may be substantially parallel to the first axis 1830 while oriented a distance away in a different plane. Thus, as the reciprocating saw assembly actuates along the first axis 1830, the plurality of cutting teeth 1888 can resect bony tissues along the cutting axis 1890 (or along a plane parallel to the cutting axis 1890), thereby forming one or more slots into the prepared tibial surface.

According to various embodiments, the saw blade 1800 may involve one or more dimensions that are similar to the previously discussed embodiments of exemplary saw blades 1000, 1200, 1500, 1700.

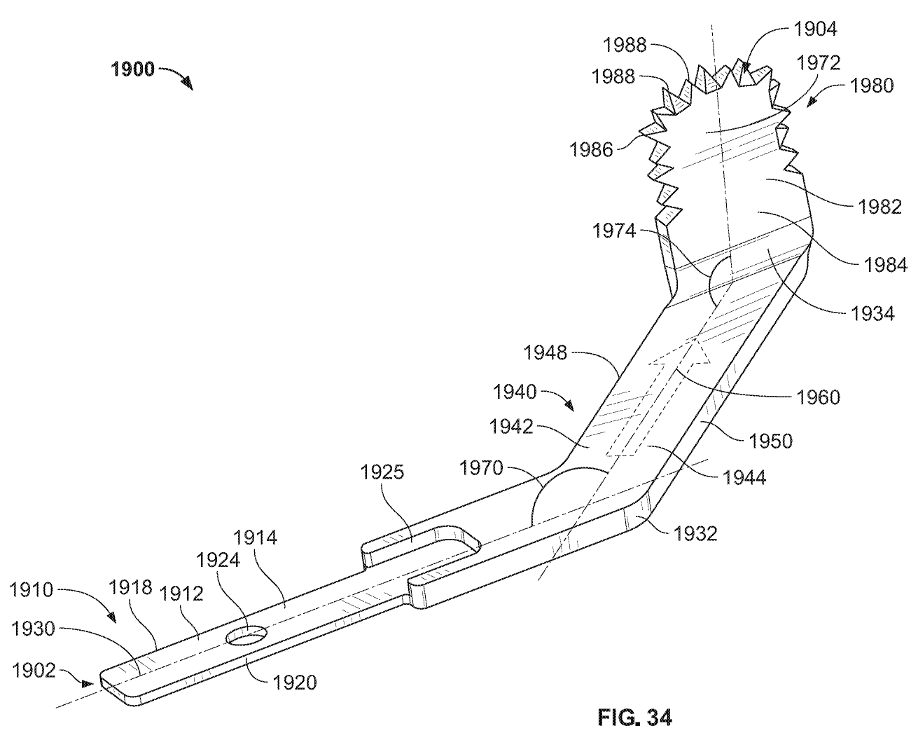

FIG. 34 shows another embodiment of an exemplary saw blade 1900 in a perspective view. Similar to the saw blade 1000 of FIGS. 1-8, saw blade 1500 of FIGS. 17-24, and saw blade 1800 of FIG. 33, this embodiment may be provided for use within a reciprocating saw during the TAR procedure. Here, the saw blade 1900 includes a first end 1902 and a second end 1904, wherein a saw interface portion 1910 is positioned at the first end 1902 and a cutting portion 1980 is positioned at the second end 1904. The saw blade 1900 includes an offset portion 1940 extending between the saw interface portion 1910 and the cutting portion 1980. In particular, this embodiment may be configured to provide an enhanced mechanical interface with a drive component of the reciprocating surgical saw and/or a powered blade collar coupled to a power source (e.g., a battery-powered motor). For instance, the saw interface portion 1910 may include a through-hole opening and an adjacent pocket configured to receive a retention feature (e.g., a pin, detent, post, fastener, or other coupling member) of the blade collar. Each feature will be described in detail below.

In some embodiments, the saw interface portion 1910 provides a temporary securement point between the saw blade 1900 (e.g., via one or more retention features) and certain receiving features of the reciprocating saw to form a reciprocating saw assembly. In some embodiments, the saw interface portion 1910 can be aligned along a first axis 1930, wherein the first axis 1930 is coaxial with the axis of reciprocation of the reciprocating saw.

In some embodiments, the first planar surface 1912 comprises a first side 1914 opposite a second side 1916, wherein the saw interface portion 1910 includes a retention feature provided in the form of a pocket 1925 recessed into the first side 1914 (or other portion of the first planar surface 1912) and/or a through-hole opening 1924 extending between the first side 1914 and the second side 1916. In some embodiments, the pocket 1925 comprises an extended groove region configured to position the through-hole opening 1924 within a comparatively more rigid section of the saw interface portion 1910 and to provide support through the pocket 1925 for increased stiffness and/or rigidity of the saw interface portion 1910 during use. In combination, the pocket 1925 and through-hole opening 1924 may provide improved retention and a more rigid engagement interface with the blade collar during high-load cutting by improving repeatable positioning and indexing of the saw blade 1900 within the blade collar, reducing relative motion during reciprocation, and improving transfer of reciprocating forces to the cutting portion 1980.

In some embodiments, the first planar surface 1912 comprises a first edge 1918 and a second edge 1920, wherein the first and second edges 1918, 1920 define lateral boundaries of the saw interface portion 1910. In some cases, a first collar engagement tab 1926 and a second collar engagement tab 1928 extend laterally or outwardly from the first edge 1918 and the second edge 1920, respectively.

In some embodiments, the offset portion 1940 can extend from the saw interface portion 1910 via an elbow bend 1932 to bridge/connect the saw interface portion 1910 with the cutting portion 1980. For instance, the elbow bend 1932 may transition the saw interface portion 1910 to the offset portion 1940 via an angle/bend by connecting the first edge 1918 with the first edge 1948 and the second edge 1920 with the second edge 1950.

In some embodiments, the offset portion 1940 can include a second planar surface 1942 having a first side 1944, a second side 1946, a first edge 1948, and a second edge 1950. In this embodiment, the second planar surface 1942 is shown as co-planar with the first planar surface 1912 while in other embodiments, the second planar surface 1942 may not be co-planar with the first planar surface 1912.

In some embodiments, the offset portion 1940 can be aligned along a second axis 1960, wherein the second axis 1960 is oriented at an offset angle 1970 relative to the first axis 1930. In this way, the offset portion 1940 is oriented at the offset angle 1970 relative to the saw interface portion 1910 via the elbow bend 1932.

In some embodiments, the cutting portion 1980 extends from offset portion 1940, the cutting portion 1980 comprising a third planar surface 1982 having a first side 1984 and a second side 1985. Here, the cutting portion 1980 further includes at least one edge 1986 having a plurality of cutting teeth 1988 disposed along the at least one edge 1986. In some cases, the plurality of cutting teeth 1988 may be disposed on a portion of the at least one edge 1986 (e.g., a top portion), while in other cases, the plurality of cutting teeth 1988 may be disposed along any suitable edge, end, or surface.

In some embodiments, the third planar surface 1982 may be aligned along a third axis 1972, wherein the third axis 1972 is oriented at a cutting angle 1974 relative to the second axis 1960. In this way, the cutting portion 1980 is oriented at the cutting angle 1974 relative to the offset portion 1940. Additionally, in some embodiments, the cutting portion 1980 may extend from the offset portion 1940 via a transition bend 1934 oriented at the cutting angle 1974, wherein the transition bend 1934 raises (or lowers) the third planar surface 1982 relative to the second planar surface 1942 to form a sloped or angular transition between the surfaces 1942, 1982.

In some embodiments, via the combination of the offset angle 1970 and the cutting angle 1974, a cutting axis 1990 may be substantially parallel to the first axis 1930 while oriented a distance away in a different plane. Thus, as the reciprocating saw assembly actuates along the first axis 1930, the plurality of cutting teeth 1988 can resect bony tissues along the cutting axis 1990 (or along a plane parallel to the cutting axis 1990), thereby forming one or more slots into the prepared tibial surface.

According to various embodiments, the saw blade 1900 may involve one or more dimensions that are similar to the previously discussed embodiments of exemplary saw blades 1000, 1200, 1500, 1700, 1800.

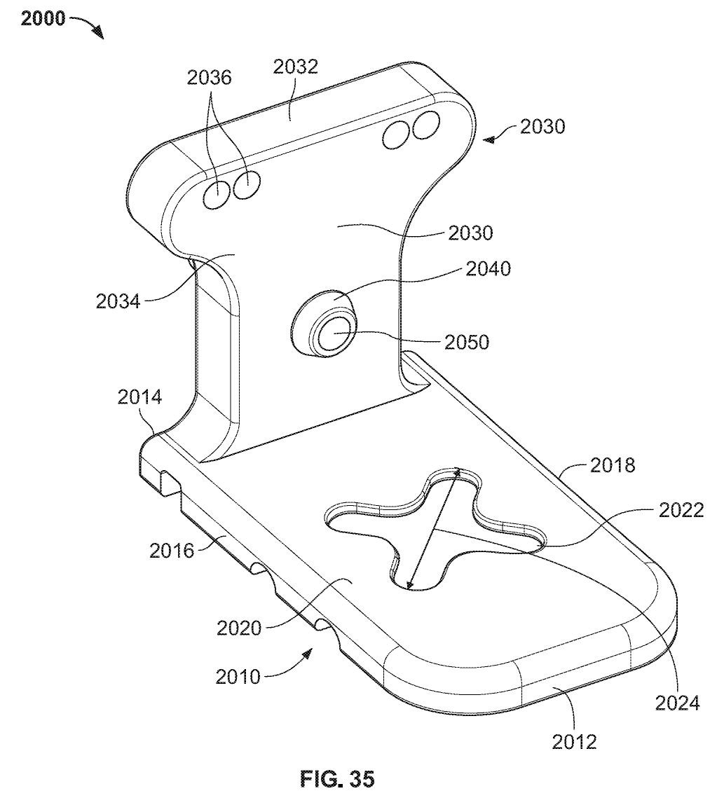

Referring now to FIG. 35, one embodiment of an exemplary tibial trial 2000 for use in the TAR procedure is shown. Generally, after preliminary resections are made to prepare the tibial surface (e.g., such that the prepared tibial surface includes one or more flat regions), the tibial trial 2000 can then be positioned on and temporarily secured to the prepared tibial surface, thereby providing a reference point for the various tools and instruments of the TAR procedure, including the exemplary saw blades 1000, 1200, 1500, 1700, 1800, 1900 discussed with reference to FIGS. 1-34. In some cases, the positioning of the tibial trial 2000 may allow for desired alignment relative to a patient’s anatomy. As will be described herein, the tibial trial 2000 may enable alignment in an anterior-posterior direction (while allowing for other degrees of freedom), and may include various features to facilitate desirable positioning and referencing for other tools of the TAR procedure. Thus, the tibial trial 2000 may provide alignment and temporary fixation for the tools of the TAR system.

In some embodiments, the tibial trial 2000 may comprise a trial base 2010—having a first end 2012, a second end 2014, a superior face 2020, an inferior face (not shown), a first side 2016, and a second side 2018—and a trial face 2030—having a top end 2032, a bottom end (not shown), an anterior face 2034, and a posterior face (not shown). In some cases, the tibial trial 2000 can be inserted into the joint space such that the posterior face engages with a portion of a patient’s tibia, and the superior face 2020, anterior face 2034, and inferior face are available to engage with tools of the TAR system. In some aspects, the trial base 2010 and the trial face 2030 may be coupled at a junction formed by the second end 2014 and the bottom end and machined together. In other aspects, the trial base 2010 and the trial face 2030 may be formed as a singular component (e.g., via 3D-printing, casting, machining, etc.).

In some embodiments, the trial base 2010 can include a superior face 2020 having various through-holes, through-slots, and other features for use with the tools and instruments of the TAR procedure. For instance, the superior face 2020 may include a reference, or access opening 2022, which forms a through-hole to enable access through the trial base 2010 to the prepared tibial space. Although the embodiment shown depicts the access opening 2022 having an X-shape, the access opening 2022 may form any suitable shape in other embodiments, such as a circular shape, ovular shape, triangular shape, quadrilateral shape, polygonal shape, or any suitable combination thereof without departing from the principles of this disclosure. In some aspects, the access opening 2022 may be generally centered on the superior face 2020 with respect to the overall trial base 2010, while in other embodiments, the access opening 2022 may be offset from the center of the superior face in the anterior-posterior direction and/or the medial-lateral direction. In some embodiments, the trial base 2010 may include any suitable number of reference or access openings disposed on any suitable portion of the superior face 2020 or any other face of the tibial trial 2000. In some embodiments, the access opening 2022 may be positioned based on patient-specific anatomy without departing from the principles of this disclosure.

In some embodiments, the access opening 2022 includes a varying diameter or cross-length 2024, measuring between about 5.0 mm to 8.0 mm, or about 7.5 mm to 10.5 mm, or about 8.0 mm to 11.0 mm, or about 10.5 mm to 13.5 mm, or about 12.0 mm to 15.0 mm, or about 13.5 mm to 16.5 mm, or about 15.0 mm to 18.0 mm, or about 16.5 mm to 19.5 mm, or about 18.0 mm to 21.0 mm, or about 20.5 mm to 23.5 mm, or about 22.0 mm to 25.0 mm, or any other suitable diameter or length. In some cases, the cross-length 2024 may vary along different portions of the access opening 2022, while in other cases, the cross-length 2024 may be static (e.g., if the access opening 2022 is circular).

In some embodiments, the cross-length 2024 may be sized to complement one or more dimensions of the cutting portions 1080, 1280, 1580, 1780 of the exemplary saw blades 1000, 1200, 1500, 1700. For instance, a user may insert the cutting portions 1080, 1280, 1580, 1780 through the access opening 2022 in the inferior-superior direction. As the reciprocating saw assembly actuates and causes a back-and-forth or oscillating motion of cutting portions 1080, 1280, 1580, 1780 parallel to the first axis (e.g., first axis 1030, 1230, 1530, 1730), the cutting portion may form cuts in the prepared tibial surface in the approximate dimensions of the cross-length 2024 and in the cutting axis (e.g., an axis co-linear to—but displaced from—the axis of reciprocation, such as cutting axis 1090, 1290, 1590, 1790, 1890, 1990).

In one non-limiting example, the user may select saw blades 1000, 1200 for use with the reciprocating saw such that saw blade 1000 forms cuts in the prepared tibial surface to approximate the dimensions of the cross-length 2024 in a first orientation, and saw blade 1200, which may be a mirror of saw blade 1000, similarly forms cuts in a second orientation. In another non-limiting example, the user may select saw blades 1500, 1700 for use within the reciprocating saw assembly such that saw blade 1500 forms cuts in the prepared tibial surface to approximate the dimensions of the cross-length 2024 in a first orientation, and saw blade 1700, which may be a mirror of saw blade 1500, similarly forms cuts in a second orientation. In other examples, any two saw blades may be used to form any suitable pattern of cuts through the access opening 2022 and into the prepared tibial surface to improve access to the medullary cavity. In additional examples, one saw blade may be used to form any suitable pattern of cuts through the access opening 2022 (e.g., access to the prepared tibial surface may allow the user to rotate the orientation of the saw blade such that multiple cuts can be made without swapping blades).

According to some embodiments, the trial face 2030 can include an anterior face 2034 having various through-holes and other features to position and retain the tibial trial 2000 in a desired orientation relative to a pre-operatively defined reference point. In some embodiments, anterior face 2034 can include a central fastener opening 2040 forming a through-hole through to the posterior face, and sized and configured to receive at least one fastener 2050. The fastener 2050 may be used to adjust and secure the positioning of the tibial trial 2000 relative to the underlying patient anatomy in an anterior-posterior direction such that tightening and loosening the fastener 2050 adjusts the tightness of the tibial trial 2000 against the patient anatomy. In this way, inserting the fastener 2050 through the central fastener opening 2040 in the anterior-posterior can temporarily fix the tibial trial 2000 to the patient anatomy. In various aspects, the fastener 2050 can be provided in the form of any suitable fastening mechanism, such as, but limited to, a nail, screw, bolt, k-wire, etc.

In some embodiments, the anterior face 2034 can include one or more apertures 2036 disposed proximate the top end 2032 forming through-holes through to the posterior face. In some cases, the one or more apertures 2036 can be sized to receive auxiliary fasteners, wherein the auxiliary fasteners prevent rotation of the tibial trial 2000 against the patient anatomy. In other cases, auxiliary fasteners can be inserted through the one or more apertures 2036 to help with positioning other tools of the TAR procedure in a desired orientation relative to the tibial trial 2000. In various aspects, auxiliary fasteners can be provided in the form of any suitable fastening mechanism, such as, but limited to, a nail, screw, bolt, k-wire, etc.

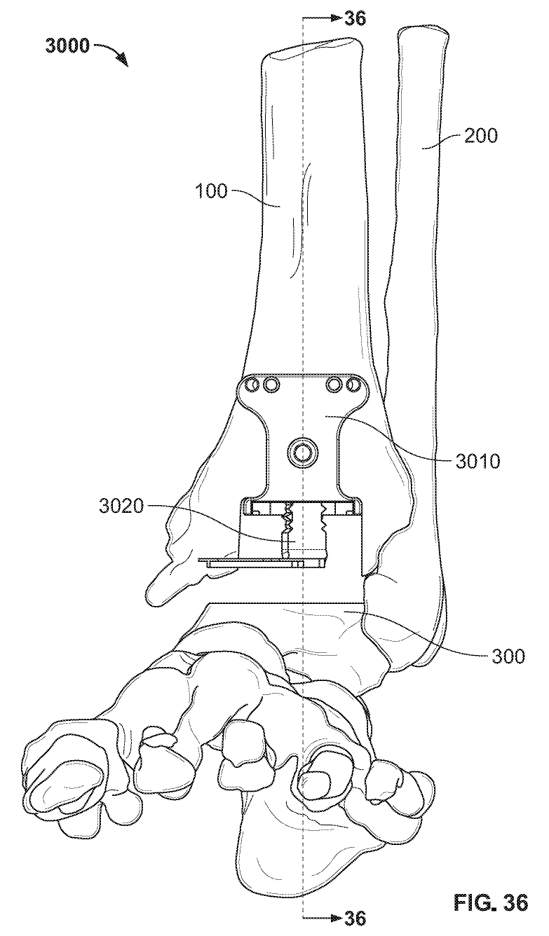

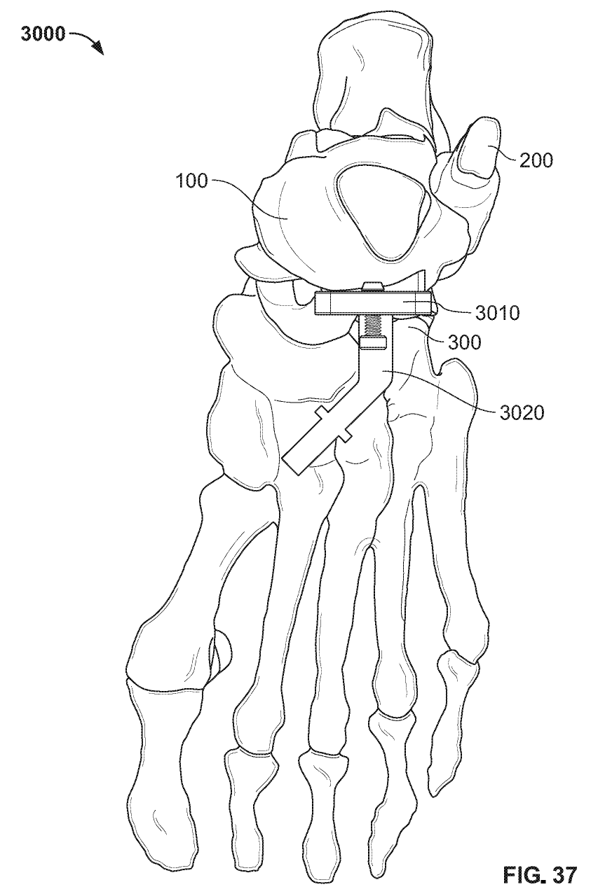

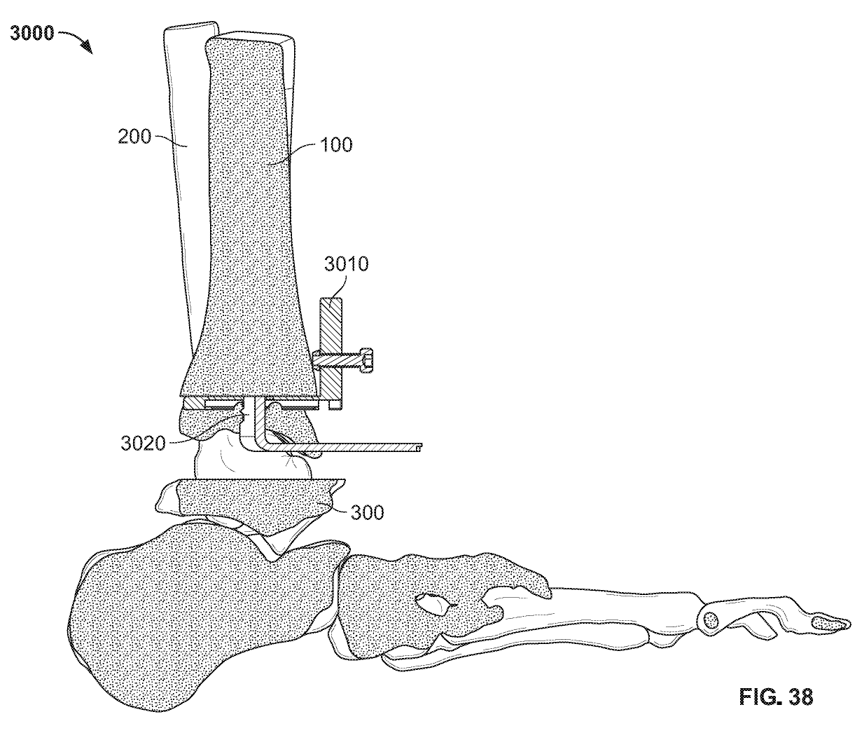

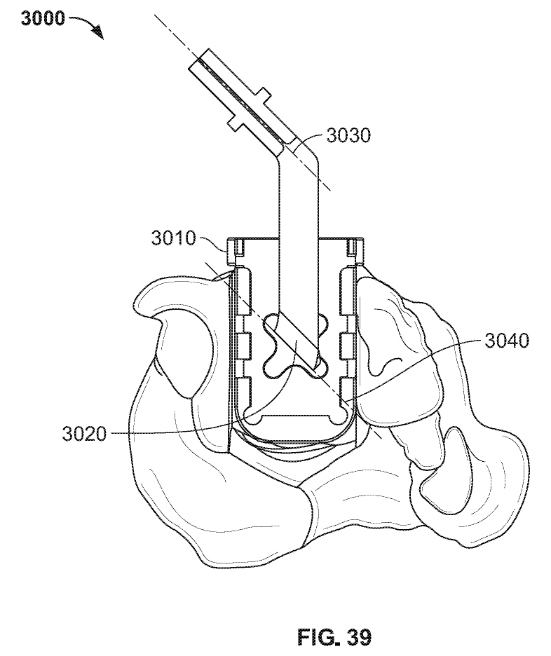

Referring now to FIGS. 36-38, one embodiment of an exemplary saw blade 3020 inserted through an exemplary tibial trial 3010 installed on a tibia is shown within a view 3000 of patient anatomy during the TAR procedure. As shown, a user has prepared the patient anatomy by resecting portions of the tibia, fibula, and talus. The tibial trial 3010 has been installed onto a desired portion of the tibia. The user may then insert the saw blade 3020 through a portion of the tibial trial 3010 (e.g., the access opening 2022) to resect a portion of the prepared tibial surface. In some cases, the saw blade 3020 may be installed or attached to a blade collar of a reciprocating saw (not shown) to form a reciprocating saw assembly. In various embodiments, the saw blade 3020 can be any of saw blades 1000, 1200, 1500, 1700, or any other suitable saw blade or combination thereof, or any other component configured for resecting bone. Similarly, the tibial trial 3010 can be tibial trial 2000 or any other suitable trial or other guide component.

Referring now to FIG. 39, an inferior view of the embodiment of FIGS. 36-38 is shown, with a talus, calcaneus, and the remaining bones of the foot hidden from view so that the saw blade 3020 positioning relative to the tibial trial 3010 can be clearly shown. Here, the reciprocating saw assembly (not shown) may cause the saw blade 3020 to form cuts through the tibial trial 3010 in a back-and-forth motion along a cutting axis 3040 (which is parallel to but displaced from first axis 3030), thereby resecting the portion of the prepared tibial surface accessible through the tibial trial 3010 (e.g., through the access opening 2022). As described herein, the first axis 3030 may be parallel with certain other features of the saw blade 3020, such as its cutting portion and associated axis of cutting. In this way, as the reciprocating saw assembly activates and moves the saw blade 3020 back-and-forth, the cutting portion of the saw blade 3020 can cut the prepared tibial surface in an axis parallel to the first axis 3030.

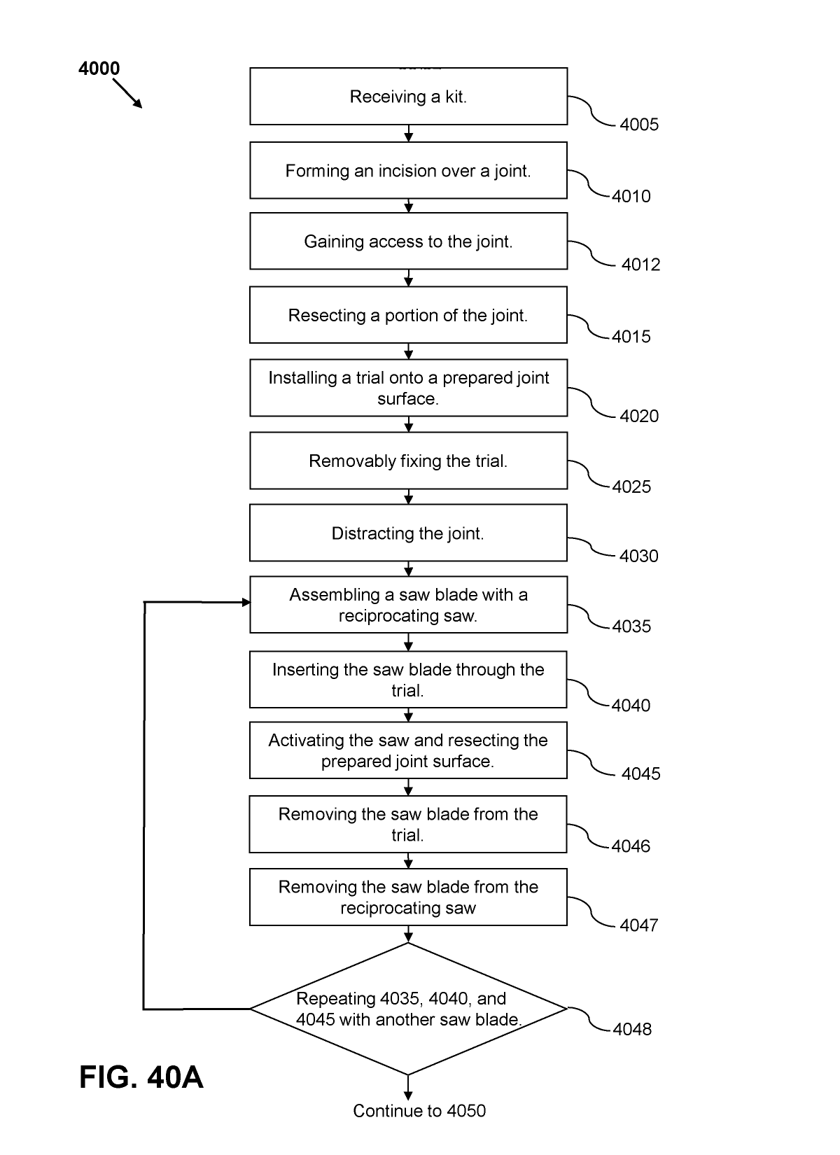

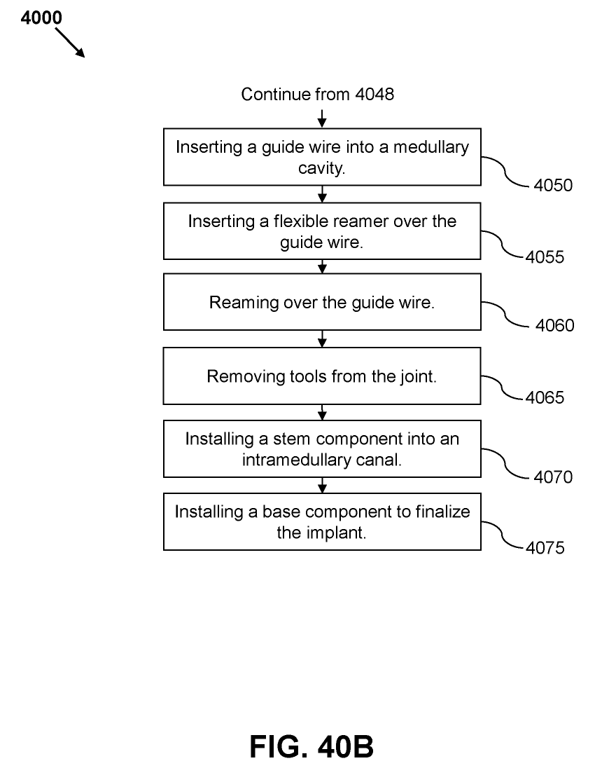

According to various embodiments, FIGS. 40A-B describes an exemplary method 4000 of preparing the tibial surface for insertion of a tibial implant. The method 4000 can comprise the following steps: (1) receiving a kit 4005; (2) forming an incision on the anterior side of an ankle joint 4010; (3) gaining access to the ankle joint 4012; (4) resecting a portion of the patient’s tibia and/or talus 4015; (5) installing a tibial trial onto a prepared tibial surface 4020; (6) removably fixing the tibial trial to the prepared tibial surface 4025; (7) distracting the ankle joint space 4030; (8) assembling the reciprocating saw assembly by inserting a first saw blade into the blade collar of a reciprocating saw 4035; (9) inserting the first saw blade through the tibial trial 4040; (10) activating the reciprocating saw to resect a portion of the prepared tibial surface, thereby forming part of a medullary cavity 4045; (11) removing the first saw blade from the tibial trial 4046; (12) removing the first saw blade from the blade collar 4047; (13) optionally repeating steps 4035, 4040, and 4045 with a second saw blade 4048; (14) inserting a guide wire into the medullary cavity 4050; (15) inserting a flexible reamer assembly over a guide wire 4055; (16) reaming over the guide wire to form an intramedullary canal 4060; (17) removing tools from the joint space 4065; (18) installing one or more stem components into the intramedullary canal 4070; and (19) installing a base component onto the one or more stem components to finalize the tibial implant 4075.

At step 4005, in some embodiments, a user (e.g., surgeon), receives or obtains a kit comprising tools, instruments, and other components suited to carry out the method 4000 by preparing the tibial surface, including, but not limited to, a tibial trial (e.g., tibial trial 2000, 3010), a distractor, a reciprocating saw assembly comprising a reciprocating saw with a blade collar and one or more saw blades (e.g., saw blades 1000, 1200, 1500, 1700, 3020), one or more guide wires (including wire guides), and a reamer (e.g., flexible reamer). Additionally, the kit may include one or more implants and other components to install within the prepared tibial surface, including at least a tibial implant.

At step 4010, in some embodiments, the user forms or makes an anterior incision on the ankle joint.

At step 4012, the user gains access to the ankle joint and its underlying bones and tissues. In particular, the user incises an anterior portion of the ankle joint to gain access to the tibia and talus from the anterior direction.

At step 4015, in some embodiments, the user resects portions of the tibia and talus to create prepared surfaces on the respective bones. In some embodiments, the prepared surfaces may be generally flat to open a desirable workspace for further tibial surface processing and to provide space for implants to be installed. In other embodiments, any other suitable surface contour may be created.

At step 4020, in some embodiments, the user installs the tibial trial onto the prepared tibial surface in a desired position (see, e.g., FIGS. 36-38). In some embodiments, certain features of the tibial trial may be used to position the trial in a desirable position, which, in turn, may function as an overall reference point for other tools of the method 4000. In other embodiments, other components, such as a sight alignment tool or other guide can be used to suitable position the tibial trial on the prepared tibial surface.

At step 4025, in some embodiments, the user removably fixes the tibial trial to a surface of the tibia (see, e.g., FIGS. 36-38). For instance, fixation can be set (1) in the anterior-posterior direction using a fastener opening (e.g., opening 2040) which is accessible from the anterior direction on a trial face (e.g., trial face 2030) of the tibial trial and a screw, bolt, or any other suitable fastener (e.g., fastener 2050); and (2) in the medial-lateral direction using apertures (e.g., apertures 2036) which are accessible from the anterior direction on the trial face and one or more guide wires, nails, pins, or any other suitable fasteners.

At step 4030, in some embodiments, the user distracts the ankle joint space to form a workspace. In some embodiments, the user can use any suitable distractor to distract the joint space, while in other embodiments, the user can distract the joint space manually. In an alternative embodiment, depending on user preference, step 4030 may be performed at a later stage in the method 4000 (e.g., after steps pertaining to using the reciprocating saw blade and before steps pertaining to reaming). For instance, steps pertaining to the reciprocating saw blade (which will be discussed in relation to steps 4035-4045) may not require the additional anterior clearance within the joint space provided by distraction, while steps pertaining to reaming (which will be discussed in relation with steps 4055 and 4060) may require additional clearance. In certain embodiments, step 4030 may be optional and thus omitted.

At step 4035, in some embodiments, the user assembles the reciprocating saw assembly. In some cases, this may involve inserting a portion of one or more saw blades (e.g., the saw interface portion) into the blade collar of the reciprocating saw. In particular, the user may insert a first saw blade into the blade collar. Further, the blade collar may be spring-loaded or otherwise configured to enable smooth selective engagement (e.g., insertion, removal) of the one or more saw blades and the reciprocating saw. Additionally, the blade collar may include various complementary features to receive certain features of the one or more saw blades (e.g., support ribs, collar engagement tabs). In some embodiments, the reciprocating saw assembly may be assembled outside of the patient, such as on a back table.

At step 4040, in some embodiments, the user positions the reciprocating saw assembly to insert the one or more saw blades through an access opening (e.g., opening 2022) of the tibial trial from the anterior direction (see, e.g., FIGS. 36-38). In particular, the user may insert the first saw blade through the tibial trial. In certain cases, the user may use a resection guide that may be removably attached to the prepared tibial surface or to the tibial trial. The resection guide may include one or more slots to guide the one or more saw blades through to the prepared tibial surface.

At step 4045, in some embodiments, the user activates the reciprocating saw to resect certain portions of the prepared tibial surface. This step involves using the one or more saw blades to begin forming an intramedullary canal in the medullary cavity of the tibia (see, e.g., FIG. 39). Specifically, the user may activate the reciprocating saw assembly to begin the reciprocating motion of the saw blade installed therein and along the axis of reciprocation. Based on the design and configuration of the saw blade (see, e.g., the discussion with reference to FIGS. 1-8), the saw blade can resect the prepared tibial surface along a cutting axis, wherein the cutting axis is parallel to but displaced from the axis of reciprocation.

At step 4046, in some embodiments, the user may remove the first saw blade from the tibial trial.

At step 4047, in some embodiments, the user may remove the reciprocating saw assembly from the tibial trial and remove the first saw blade from the blade collar.

At step 4048, in some embodiments, steps 4035, 4040, and 4045 may optionally be repeated using a second saw blade. For instance, using the first saw blade, the user may resect a first pattern of cuts (e.g., in a partial X-shape slot pattern) through the access opening to begin forming the intramedullary canal. The user may then repeat steps 4035, 4040, and 4045 using a second saw blade with the reciprocating saw (which may be an altered or mirrored form of the initial reciprocating saw assembly) to resect a second pattern of cuts (e.g., in a complementary and partial X-shape slot pattern) to continue forming the intramedullary canal.

In one non-limiting example, the first saw blade may form the first pattern of cuts in a first direction via the reciprocating motion and the superiorly-oriented force provided by the assembly until the first saw blade bottoms out or the user decides to stop. Then, the user may remove the first saw blade from the assembly and replace it with the second saw blade to form a second pattern of cuts in a second direction until the second saw blade bottoms out or the user decides to stop. In this way, a full pattern of cuts (e.g., X-shape) may be completed. Thus, the user may have improved flexibility in targeting specific patterns of bone for resection while reducing user judgment.

In certain embodiments, the user may additionally impact the prepared tibial surface at the site of the full pattern of cuts. In this way, the user may increase access into the medullary cavity. In some cases, this optional step may be performed with a broach or any other suitable impacting tool. In other embodiments, both the first and the second saw blades may be inserted simultaneously to perform steps 4035, 4040, 4045.

At step 4050, in some embodiments, the user inserts a guide wire into the medullary cavity accessible via the full pattern of cuts formed in step 4045. In some cases, the user may also use a wire guide or other guiding instrument to enable the guide wire to bend into the joint space from the anterior direction while maintaining suitable vertical alignment. In certain cases, the user may monitor progression of the guide wire into the medullary cavity under X-ray or other fluoroscopic imaging.

At step 4055, in some embodiments, the user inserts a flexible reamer assembly (including, at least, a reamer blade, a flexible reamer, a reamer guide, a reamer lock plate, etc.) into the tibial trial from the anterior direction. To do so, the user may first slide a hollow central channel of the reamer blade (which is in fluid communication with the flexible reamer) over the guide wire and then insert the rest of the flexible reamer assembly into the tibial trial such that the reamer blade is positioned at the access opening. In some embodiments, the flexible reamer assembly may be assembled outside of the patient, such as on a back table.

At step 4060, in some embodiments, the user reams over the guide wire to widen the medullary canal and form an intramedullary canal sized to a predetermined and/or patient-specific distance. In some embodiments, the guide wire and the reamer blade may be driven independently (e.g., one after another), while in other embodiments, the guide wire and the reamer blade may be driven in parallel (e.g., in a step-wise manner). In certain cases, the user may monitor progression of the reamer blade into the medullary cavity under X-ray or other fluoroscopic imaging.

At step 4065, in some embodiments, the user removes one or more tools and instruments from the patient while leaving the tibial trial and distractor (if used) in place. In some embodiments, the user may also remove the distractor if used.

At step 4070, in some embodiments, the user installs or inserts one or more stem components of the tibial implant into the intramedullary canal.

At step 4075, in some embodiments, the user installs or attaches a base component of the tibial implant to the one or more stem components. The user may then finalize installation of the tibial implant and remove the tibial trial and any other tools remaining in the joint space.

One skilled in the art will understand that processes discussed herein can include machine learning processes and other advanced artificial intelligence processes. For example, the system and processes of the present disclosure can perform diagnostics, image analysis, generate tasks or action items, provide customized recommendations according to user settings and preferences, generate 3D device models, generate surgical plans, generate computer-aided design (CAD) files, generate operating instructions for a surgical robot, generate personalized implant designs, generate notifications, and similar processes. Further, in at least one embodiment, the systems and processes discussed herein may provide an input to an additional process (e.g., surgery performed by a robot) or may be part of another process. In some embodiments, the system may use additional inputs and/or feedback loops to an iterative training process for a personalized implant generation process based on a plurality of parameters and adjustable metric values. Examples of such processes can be found in U.S. Patent Appln. No. 18/454,580, filed Aug. 23, 2023, and entitled “PATIENT-SPECIFIC MEDICAL DEVICES AND ADDITIVE MANUFACTURING PROCESSES FOR PRODUCING THE SAME”, incorporated by reference herein in its entirety.

In some embodiments, the system can include a process for generating customized surgical plans that are populated for approval by a medical provider, surgeon, or similar healthcare professional. In various embodiments, the customized surgical plans can include one or more operating instructions to be transmitted to, installed on, or otherwise executed by a surgical robot or similar device. In some embodiments, the surgical robot can be autonomous or semi-autonomous.

As an example, implants, instruments, surgical plans, techniques, or information related to implants, instruments, surgical plans, or techniques, may be downloaded, saved, or otherwise captured within a file for use with a surgical robot, navigation system, or in another surgical setting. In at least one embodiment, the surgical plan may be transmitted to a surgical robot, navigation system, or other operating room system or device via a wireless transmission system (e.g., cellular, wifi, or other data transfer mechanism). Some non-limiting examples of file types that may be generated and/or transmitted include, but are not limited to: DICOM, STL, OBJ, TIFF, BMP, JPEG, AMF, 3MF, PLY, FBX, STEP/IGES, NIfTI, PDF, DOCX, CSV, .PRT, .ASM, .SLDPRT, .SLDASM, .PLAN, .NWD, .NWF, VRML, MIMICS, .DWG, etc.

In one non-limiting embodiment, the surgical robot can include a controller, a processor, a memory unit, a surgery module, a vision system (and/or other navigation systems), a sensor module, and a communication module. In various embodiments, the surgical robot may include a processor designed to execute patient-specific instructions to the surgery module to perform various actions associated with a surgical procedure. In some embodiments, the controller is designed to communicate with the processor and the memory unit to execute instructions to control one or more aspects of the surgery module. In some embodiments, the controller can be provided in the form of a virtual reality (VR) and/or augmented reality (AR) headset or similar controller. In some embodiments, the surgery module can include one or more articulating arms, one or more surgical devices (e.g., scalpel, retractor, drill, saw, suture instrument, suction, electrocautery, etc.), attachment(s) for accessories, or a combination thereof. In some embodiments, the surgical robot can also include a vision system used to track the movement of one or more aspects of the surgery module, a patient, a surgeon, the surgical environment, etc. As will be understood, the surgical robot may include or be in communication with other navigation systems, including, but not limited to, radar or sensor-based navigation (e.g., Bluetooth or BLE) and such instructions discussed herein may include instructions on navigation via one or more navigation systems. In various embodiments the surgical robot can utilize the sensor module to receive feedback signals related to the surgical operations or other actions performed by the surgical robot, patient, surgeon, or environment to compensate for changes during surgery or to otherwise facilitate execution of a surgical plan. In some embodiments, the sensor module is operatively coupled to the controller of the surgical robot. The communication module can be used to send instructions, feedback, signals, or other data between one or more aspects of the surgical robot, the surgeon, a computing system, or a combination thereof. In some embodiments, the communication module can be designed to generate notifications, transmit updated imaging, initiate safety alerts, send signals to particular surgical devices to indicate an implant is properly (or improperly) aligned, or similar data related to various actions or tasks being executed by the surgical robot, the surgeon, the medical team, or other aspects of the system or processes described herein.

In various embodiments, exemplary systems may receive information or feedback from a surgical robot or navigation system and update a machine learning process, one or more emphasis guidelines, weighting factors, or like processes or parameters to create a feedback loop to improve an existing surgical plan or a future surgical plan.

### Conclusion

While the particular embodiments described herein relate to ankle replacement-based applications, it will be recognized and appreciated that the systems and processes described herein may be applicable to other joint implant-based applications. It will be appreciated by one skilled in the art that this embodiment is only a non-limiting example used to illustrate that the system and processes are designed to install an ankle implant that has patient specific-geometry and a patient specific surgical plan.

Additional aspects, features, and processes of the claimed systems, devices, and processes will be readily discernible from the description herein, by those of ordinary skill in the art. Many embodiments and adaptations of the disclosure other than those herein described, as well as many variations, modifications, and equivalent arrangements and processes, will be apparent from or reasonably suggested by the disclosure and the description thereof, without departing from the substance or scope of the claims. Furthermore, any sequence(s) and/or temporal order of steps of various processes described and claimed herein are those considered to be the best mode contemplated for carrying out the claimed systems. It should also be understood that, although steps of various processes may be shown and described as being in a preferred sequence or temporal order, the steps of any such processes are not limited to being carried out in any particular sequence or order, absent a specific indication of such to achieve a particular intended result. In most cases, the steps of such processes may be carried out in a variety of different sequences and orders, while still falling within the scope of the claimed systems. In addition, some steps may be carried out simultaneously, contemporaneously, or in synchronization with other steps.

Aspects, features, and benefits of the claimed devices and processes for using the same will become apparent from the information disclosed in the other applications as incorporated by reference. Variations and modifications to the disclosed systems and processes may be affected without departing from the spirit and scope of the novel concepts of the disclosure.

It will, nevertheless, be understood that no limitation of the scope of the disclosure is intended by the information disclosed in the applications incorporated by reference; any alterations and further modifications of the described or illustrated embodiments, and any further applications of the principles of the disclosure as illustrated therein are contemplated as would normally occur to one skilled in the art to which the disclosure relates. The description of the disclosed embodiments has been for illustration and description and is not intended to be exhaustive or to limit the devices and processes for using the same to the precise forms disclosed. Many modifications and variations are possible in light of the above teaching.

The embodiments were chosen and described to explain the principles of the devices and processes for using the same and their practical application to enable others skilled in the art to utilize the devices and processes for using the same and various embodiments and with various modifications as are suited to the particular use contemplated. Alternative embodiments will become apparent to those skilled in the art to which the present devices and processes for using the same pertain without departing from their spirit and scope. Accordingly, the scope of the present devices and processes for using the same is defined by the appended claims rather than the description and the embodiments described therein.

What is claimed is:

1. A saw blade for a reciprocating surgical saw, the reciprocating surgical saw having an axis of reciprocation, the saw blade comprising:
a first end and a second end;
a saw interface portion at the first end, the saw interface portion comprising:
a first surface;
a first edge;
a second edge; and
a first axis,
wherein the first axis is coaxial with an axis of reciprocation of a reciprocating surgical saw,
wherein the saw interface portion is configured to be removably coupled to a drive component of the reciprocating surgical saw;
an offset portion extending from the first edge, wherein the offset portion extends from the first edge at an offset angle between about 90° and 180° relative to the first edge;
the offset portion comprising a second surface, the second surface substantially co-planar with the first surface;
a cutting portion extending from the offset portion, wherein the cutting portion comprises a third surface, the third surface extending at a cutting angle between about 0° and 90° relative to the second surface; and
a plurality of cutting teeth disposed along at least one edge of the cutting portion,
wherein the third surface is substantially parallel to the axis of reciprocation.

2. The saw blade of claim 1, wherein the saw blade comprises a metallic material.

3. The saw blade of claim 1, further comprising at a first collar engagement tab extending from the first edge of the saw interface portion.

4. The saw blade of claim 3, further comprising a second collar engagement tab extending from the second edge of the saw interface portion.

5. The saw blade of claim 1, wherein the saw interface portion further comprises a retention feature.

6. The saw blade of claim 5, wherein the retention feature is a pocket that is recessed into the first surface.

7. The saw blade of claim 5, wherein the retention feature is a first support rib extending perpendicularly from a first side of the first surface.

8. The saw blade of claim 7, wherein the retention feature further comprises a second support rib extending perpendicularly from a second side of the first surface.

9. The saw blade of claim 5, wherein the retention feature is a through-hole opening.

10. A method for resecting bone tissue, the method comprising:
making an incision to access a patient’s bone tissue;
obtaining a reciprocating surgical saw, the reciprocating surgical saw comprising:
an axis of reciprocation; and
a blade collar;
obtaining a saw blade for the reciprocating surgical saw, the saw blade comprising:
a saw interface portion comprising:
a first planar surface;
a first edge;
a second edge; and
a first axis that is coaxial with the axis of reciprocation;
an offset portion extending from the first edge, the offset portion comprising a second planar surface, the second planar surface being co-planar with the first planar surface,
wherein the offset portion extends from the first edge at an offset angle between about 90° and 180° relative to the first edge;
a cutting portion extending from the offset portion, wherein the cutting portion comprises a third planar surface, the third planar surface extending at a cutting angle between about 0° and 90° relative to the second planar surface;
a plurality of cutting teeth disposed along at least one edge of the cutting portion; and
wherein the third planar surface is substantially parallel to the axis of reciprocation;
inserting the saw interface portion into the blade collar and securing the saw blade to the reciprocating surgical saw; and
resecting a portion of the bone tissue using the reciprocating surgical saw.

11. The method of claim 10, further comprising the steps of:
positioning a resection guide on a bone surface, the resection guide including a receiving feature for guiding the saw blade;
inserting the saw blade through the receiving feature; and
activating the reciprocating surgical saw.

12. The method of claim 11, wherein the receiving feature is a blade collar.

13. The method of claim 11, wherein the step of positioning the resection guide on the bone surface further includes removably fixing the resection guide to the bone surface.

14. The method of claim 10, wherein the saw blade further comprises a retention feature disposed on the first planar surface.

15. The method of claim 14, wherein the retention feature is provided in form of at least one support rib, the at least one support rib extending perpendicularly from at least one side of the first planar surface.

16. The method of claim 14, wherein the retention feature is provided in form of a through-hole opening disposed on a portion of the first planar surface.

17. The method of claim 14, wherein the retention feature is provided in form of a pocket that is recessed onto a portion of the first planar surface.

18. A method for total ankle replacement (TAR) of an ankle of a patient, wherein the ankle includes a tibia having a medullary cavity, the method comprising:
making an anterior incision to the ankle;
resecting a portion of the tibia to form a prepared tibial surface;
positioning a tibial trial on the prepared tibial surface;
removably fixing the tibial trial to the tibia;
distracting the ankle to form a workspace within the ankle;
inserting a saw blade into the tibial trial, the saw blade removably attached to a reciprocating surgical saw, the saw blade comprising:
a saw interface portion comprising:
a first surface;
a first edge;
a second edge; and
a first axis,
wherein the first axis is coaxial with an axis of reciprocation of a reciprocating surgical saw,
wherein the saw interface portion is configured to be removably coupled to a drive component of the reciprocating surgical saw;
an offset portion extending from the first edge, the offset portion comprising a second planar surface, the second planar surface being co-planar with the first planar surface, wherein the offset portion extends from the first edge at an offset angle between about 90° and 180° relative to the first edge;
a cutting portion extending from the offset portion, wherein the cutting portion comprises a third planar surface, the third planar surface extending at a cutting angle between about 0° and 90° relative to the second planar surface;
a plurality of cutting teeth disposed along at least one edge of the cutting portion,
wherein the third planar surface is substantially parallel to the axis of reciprocation, and
the reciprocating surgical saw comprising:
an axis of reciprocation; and
a blade collar;
resecting the prepared tibial surface with the saw blade to form an intramedullary canal;
inserting a guide wire into the medullary cavity;
inserting a flexible reamer assembly into the tibial trial;
reaming into the medullary cavity to continue forming the intramedullary canal;
inserting a stem component of a tibial implant into the intramedullary canal; and
attaching a base component of the tibial implant to the stem component.

19. The method of claim 18, further comprising the steps of:
positioning a resection guide in the tibial trial, the resection guide including a retention feature for guiding the saw blade;
inserting the saw blade through the retention feature; and
activating the reciprocating surgical saw.

20. The method of claim 18, wherein the step of positioning the resection guide in the tibial trial further includes removably securing the resection guide to the tibial trial.
